(19) United States
(12) Patent Application Publication
(10) Pub. No.: US 2024/0026431 A1
(43) Pub. Date: Jan. 25, 2024

(54) Compositions and Methods for Assessing DNA Damage in a Library and Normalizing Amplicon Size Bias

(71) Applicant: Illumina, Inc., San Diego, CA (US)

(72) Inventors: Andrew B. Kennedy, San Diego, CA (US); Lena Storms, San Diego, CA (US); Fei Shen, San Diego, CA (US); Olivia Benice, San Diego, CA (US); Eric Murtfeldt, San Diego, CA (US); Kaitlin Pugliese, San Diego, CA (US); Michael Howard, San Diego (CA)

(73) Assignee: Illumina, Inc., San Diego, CA (US)

(21) Appl. No.: 18/473,564

(22) Filed: Sep. 25, 2023

Related U.S. Application Data
(63) Continuation of application No. PCT/US22/22184, filed on Mar. 28, 2022.
(60) Provisional application No. 63/227,550, filed on Jul. 30, 2021.
(60) Provisional application No. 63/167,171, filed on Mar. 29, 2021.

(51) Int. Cl. C12Q 1/6844

(52) U.S. Cl. CPC C12Q 1/6844; C12Q 2600/166; C12Q 2600/156

(57) ABSTRACT

Described herein are standards and methods of normalizing amplicon size bias. These standards may comprise unique molecular identifiers. In some embodiments, the standards and methods are for use with next generation sequencing (NGS) assays. Also described herein are methods for quantifying DNA damage in a sample comprising DNA using fluorescence or for determining the presence of DNA damage in a library.

### CROSS-REFERENCE TO RELATED APPLICATIONS

This application is a bypass continuation of PCT/US2022/022184, filed Mar. 28, 2022, which claims the benefit of priority of U.S. Provisional Application No. 63/167,171, filed Mar. 29, 2021, and Application No. 63/227,550, filed Jul. 30, 2021; each of which is incorporated by reference herein in its entirety for any purpose.

### DESCRIPTION

### Field

This application relates to standards and methods for assessing library damage and normalizing amplicon size bias in next generation sequencing (NGS) assays. This application also relates to quantifying DNA damage in a sample comprising DNA using fluorescence.

### Background

Common methods to detect and quantify large insertion/deletion variants (indels) in genome-editing or oncology applications involve a targeted “long amplicon” PCR (LongAmp, greater than 1 kb) followed by long-read sequencing or conversion to short-read libraries for (shortread) NGS. Size-based biases in “long” PCR amplification complicate the process of accurately quantifying the relative frequency of large indel variants, however. Strategies tagging the ends of target DNA molecules with unique molecular indices prior or during amplification require the variant and UMI identified in the same NGS read. Accordingly, tagging methods with long amplicon libraries require long-read sequencing or complicated synthetic long read library prep. The post-amplification library conversion step for short read NGS makes this UMI-end tagging inappropriate, as short read NGS could decouple variant sequence and original amplicon UMIs into separate reads.

These present methods incorporate short-read NGS with UMI-containing synthetic DNA controls of varying length for normalizing amplicon size bias. The DNA controls are designed such that the identity of the standard and the UMI will be contained in same NGS read. Running control assays with these standards or spiking-in a known amount of these standards into each LongAmp assay enables bioinformatic analysis of sized-based PCR biases and facilitates better estimates of the frequency of large indels by accounting for the quantified PCR size biases.

Another issue with libraries for long-read sequencing (i.e., long-read libraries) is the presence of damaged library molecules. Assessment of the quality of long-read library preparations could be used to predict the success of subsequent workflow steps and sequencing. Long library molecules can be easily nicked or damaged during standard workflows, resulting in a library molecule that is unassociated with an adapter sequence and therefore cannot be used in workflows requiring adapters, such as sequencing. Library preparation steps can damage the DNA, either by pipetting, storage, or other handling and/or technique errors. If nicked DNA passes through a library preparation that requires both a 5′ and a 3′ adapter, the nicked DNA will be unusable in downstream steps. Library damage that is not accounted for can thus cause inaccurate estimates of library concentrations, poor sequencing coverage, and overall poor sequencing assay metrics.

A library quality control (QC) method to accurately quantify the undamaged library molecules in a library preparation could help resolve this issue. The quantitative PCR (qPCR) QC method described herein assesses library preparation quality to avoid proceeding in subsequent workflow steps with inaccurate concentrations of library. These methods can thus avoid loss of user time, money, and reagents and other consumables.

Further, DNA damage from the environment, preparation and treatment of samples, or storage conditions can significantly affect the consistency of library preparation quality. For example, during the sequencing process, the accumulation of DNA damage from exposure to low-wavelength lasers and other chemicals during sequencing cycles can increases the error rate of sequencing. A user may wish to evaluate this damage. Described herein is a method of quantifying DNA damage using fluorescence. Other assays developed to quantify DNA damage using fluorescence (such as US 2014/0030705, WO 2010028388, and US 20090042205) have been hampered by low signal-to-noise ratios, likely in part due to nonspecific binding of unincorporated fluorescent nucleotides. The present method of measuring DNA damage incorporates steps of dephosphorylation of dNTPs and of binding/elution of repaired DNA from carboxylate or cellulose beads to improve the signal and allow for a greater dynamic range of the assay.

### SUMMARY

Described herein is a pool of nucleic acid standards of different lengths, wherein the nucleic acid standards comprise a unique molecular identifier (UMI) and a 5′ universal oligonucleotide, wherein the 5′ universal oligonucleotide is the same for all standards; a 3′ universal oligonucleotide, wherein the 3′ universal oligonucleotide is the same for all standards; and at least one region between the UMI and the 5′ universal oligonucleotide and/or between the UMI and the 3′ universal oligonucleotide; wherein the length of the at least one region(s) determines the length of the standard. Also described herein are methods of quality control of libraries.

Embodiment 1. A pool of nucleic acid standards of different lengths, wherein the nucleic acid standards comprise a unique molecular identifier (UMI) and:
a. a 5′ universal oligonucleotide, wherein the 5′ universal oligonucleotide is the same for all standards;
b. a 3′ universal oligonucleotide, wherein the 3′ universal oligonucleotide is the same for all standards; and
c. at least one region between the UMI and the 5′ universal oligonucleotide and/or between the UMI and the 3′ universal oligonucleotide;
   wherein the length of the at least one region determines the length of the standard.

Embodiment 2. The pool of standards of embodiment 1, wherein the pool further comprises a further nucleic acid standard that comprises a UMI and:
a. a 5′ universal oligonucleotide, wherein the 5′ universal oligonucleotide is the same for all standards; and
b. a 3′ universal oligonucleotide, wherein the 3′ universal oligonucleotide is the same for all standards;
   wherein the further nucleic acid standard does not comprise at least one region between the UMI and the 5′ universal oligonucleotide or between the UMI and the 3′ universal oligonucleotide.

Embodiment 3. The pool of standards of embodiment 1, wherein the at least one region between the UMI and the 5′ universal oligonucleotide and/or between the UMI and the 3′ universal oligonucleotide comprise 0.2 kb-10 kb.

Embodiment 4. The pool of standards of any one of embodiments 1-3, wherein the 5′ universal oligonucleotide and/or the 3′ universal oligonucleotide each comprise an amplicon amplified from a sequence of interest.

Embodiment 5. The pool of standards of any one of embodiments 1 or 3-4, wherein the at least one region between the UMI and the 5′ universal oligonucleotide and/or between the UMI and the 3′ universal oligonucleotide each comprise an amplicon amplified from a sequence of interest.

Embodiment 6. The pool of standards of any one of embodiments 1 or 3-5, wherein the least one region between the UMI and the 5′ universal oligonucleotide and/or between the UMI and the 3′ universal oligonucleotide each comprise an arbitrary sequence.

Embodiment 7. A pool of nucleic acid standards of different lengths, wherein the nucleic acid standards comprise a UMI and:
a. a 5′ partially overlapping oligonucleotide, wherein the 5′ partially overlapping oligonucleotide is identical over at least a portion of its sequence for all the standards; and/or
b. a 3′ partially overlapping oligonucleotide, wherein the 3′ partially overlapping oligonucleotide is identical over at least a portion of its sequence for all the standards;
   wherein the lengths of the 5′ partially overlapping oligonucleotide and/or the 3′ partially overlapping oligonucleotide determines the length of the standard.

Embodiment 8. The pool of standards of embodiment 7, wherein:
a. the 5′ partially overlapping oligonucleotide comprises at least a first portion of a sequence of interest; and
b. the 3′ partially overlapping oligonucleotide comprises at least a second portion of a sequence of interest.

Embodiment 9. The pool of standards of any one of embodiments 7-8, wherein the 5′ partially overlapping oligonucleotide and/or the 3′ partially overlapping oligonucleotide each comprise a sequence that is 20 bp-1 kb smaller than a sequence of interest.

Embodiment 10. The pool of standards of any one of embodiments 7-9, wherein the 5′ partially overlapping oligonucleotide and/or the 3′ partially overlapping oligonucleotide each comprise an amplicon amplified from a sequence of interest.

Embodiment 11. The pool of standards of any one of embodiments 1-10, wherein the standards comprise double-stranded nucleic acid.

Embodiment 12. The pool of standards of any one of embodiments 1-11, wherein the standards comprise double-stranded DNA.

Embodiment 13. The pool of standards of any one of embodiments 1-12, wherein each standard comprises a different UMI.

Embodiment 14. The pool of standards of any one of embodiments 1-13, wherein the UMIs comprised in the pool of standards are a random set of sequences comprising 16-20 base pairs.

Embodiment 15. The pool of standards of embodiment 14, wherein the UMIs comprised in the pool of standards are a random set of sequences comprising 18 base pairs.

Embodiment 16. The pool of standards of any one of embodiments 1-15, wherein the pool of standards comprises 1×10¹⁰ or greater, 10×10¹⁰ or greater, or 100×10¹⁰ or greater standards, wherein each standard comprises a different UMI.

Embodiment 17. The pool of standards of any one of embodiments 1-16, wherein the number of standards in the pool is greater than the number of amplicons generated by an amplification reaction.

Embodiment 18. A pool of standards, wherein at least a first portion of the standards are from any one of embodiments 1-6 or 11-17 and wherein at least a second portion of the standards are from any one of embodiments 7-17.

Embodiment 19. A method of generating a pool of nucleic acid standards comprising:
a. providing multiple copies of at least one sequence of interest comprising nucleic acids;
b. providing a collection of oligonucleotides each comprising a UMI;
c. providing a collection of insertion oligonucleotides of varying lengths; and
d. ligating at least one sequence of interest of (a), at least one oligonucleotide comprising a UMI of (b), and at least one insertion amplicon of (c) to produce multiple nucleic acid standards of the pool of nucleic acid standards.

Embodiment 20. The method of embodiment 19, wherein the at least one sequence of interest and/or insertion oligonucleotide are prepared by amplification.

Embodiment 21. The method of embodiment 19 or embodiment 20, wherein the sequence of interest, the oligonucleotides each comprising a UMI, and/or the insertion oligonucleotides comprise a restriction enzyme cleavage site.

Embodiment 22. The method of embodiment 21, wherein the restriction enzyme cleavage site is proximal to the 5′ and/or 3′ end of the sequence of interest, the oligonucleotides each comprising a UMI, and/or the insertion oligonucleotides.

Embodiment 23. The method of embodiment 21 or embodiment 22, wherein the method further comprises cleaving the sequence of interest, the oligonucleotides each comprising a UMI, and/or the insertion oligonucleotides with a restriction enzyme before the ligating.

Embodiment 24. The method of embodiment 23, wherein the cleaving with a restriction enzyme produces sticky ends for the ligating.

Embodiment 25. A method of generating a pool of nucleic acid standards comprising:
a. providing multiple copies of at least one sequence of interest comprising nucleic acids;
b. providing a collection of oligonucleotides each comprising a UMI; and
c. ligating at least one sequence of interest of (a) and at least one oligonucleotide comprising a UMI of (b).

Embodiment 26. The method of embodiment 25, wherein the at least one sequence of interest are prepared by amplification.

Embodiment 27. The method of embodiment 25 or 26, wherein the sequence of interest and/or the oligonucleotides each comprising a UMI comprise a restriction enzyme cleavage site.

Embodiment 28. The method of embodiment 27, wherein the restriction enzyme cleavage site is proximal to the 5′ and/or 3′ end of the sequence of interest and/or the oligonucleotides each comprising a UMI.

Embodiment 29. The method of embodiment 27-28, wherein the method further comprises cleaving the sequence of interest and/or the oligonucleotides each comprising a UMI with a restriction enzyme before the ligating.

Embodiment 30. The method of embodiment 29, wherein the cleaving with a restriction enzyme produces sticky ends for the ligating.

Embodiment 31. A method of normalizing amplicon size bias comprising:
a. combining a sample comprising a target nucleic acid with a pool of nucleic acid standards of different lengths, wherein each standard comprises a UMI;
b. amplifying the standards and amplicons of a sequence of interest comprised in the target nucleic acid;
c. sequencing the standards and the amplicons of the sequence of interest to generate sequencing data;
d. determining a bias profile based on amplicon size using sequencing data from the standards; and
e. normalizing amplicon size bias using the bias profile.

Embodiment 32. The method of embodiment 31, wherein the standards in the pool of nucleic acid standards range from 0.2 kb to 20 kb base pairs.

Embodiment 33. The method of embodiment 31 or embodiment 32, wherein each standard comprised in the pool of nucleic acid standards comprises a different a UMI.

Embodiment 34. The method of embodiment 31-33, wherein the UMIs comprised in the pool of standards are a random set of sequences comprising 16-20 base pairs.

Embodiment 35. The method of embodiment 31-34, wherein the UMIs comprised in the pool of standards are a random set of sequences comprising 18 base pairs.

Embodiment 36. The method of any one of embodiments 31-35, wherein the pool of standards comprises 1×10¹⁰ or greater, 10×10¹⁰ or greater, or 100×10¹⁰ or greater standards, wherein each standard comprises a different UMI.

Embodiment 37. The method of any one of embodiments 31-36, wherein the number of standards in the pool of standards is greater than the number of amplicons generated by the amplifying.

Embodiment 38. The method of any one of embodiments 31-37, wherein the pool of nucleic acid standards comprises the pool of nucleic acid standards of any one of embodiments 1-18.

Embodiment 39. The method of any one of embodiments 31-37, wherein the pool of nucleic acid standards comprises a first portion comprising the pool of nucleic acid standards of any one of embodiments 1-6 or 11-17 and a second portion comprising the pool of nucleic acid standards of any one of embodiments 7-17.

Embodiment 40. The method of any one of embodiments 31-39, wherein the sequence of interest comprises a restriction enzyme cleavage site that is not at or in close proximity to the 5′ and/or 3′ end of the sequence of interest.

Embodiment 41. The method of any one of embodiments 31-40, wherein the sequence of interest may comprise insertion or deletion mutations.

Embodiment 42. The method of any one of embodiments 31-41, wherein the sequence of interest has been subjected to gene editing, optionally wherein the sequence of interest comprises a cut site introduced by gene editing.

Embodiment 43. The method of any one of embodiments 31-42, wherein amplifying amplicons of the sequence of interest comprises amplifying amplicons from the target nucleic acid with a pair of PCR primers that bind to primer binding sequences at the ends of the sequence of interest.

Embodiment 44. The method of any one of embodiments 31-43, wherein the standards comprise the same primer binding sequences as those at the ends of the sequence of interest.

Embodiment 45. The method of any one of embodiments 31-44, further comprising generating a library of fragments after the amplifying and before the sequencing.

Embodiment 46. The method of embodiment 31-45, wherein the generating a library of fragments is by tagmentation.

Embodiment 47. The method of any one of embodiments 31-46, wherein the sequencing data from the standards used to determine the bias profile is the unique molecule count of UMIs comprised in the standards.

Embodiment 48. A method of determining the presence of DNA damage in a library comprising one or more library molecule, wherein each library molecule comprises a double-stranded DNA insert with a hairpin adapter at each end of the insert, comprising:
a. denaturing the first stand and second strand of the double-stranded DNA inserts comprised in library molecules;
b. annealing a forward primer and a reverse primer to library molecules;
c. amplifying to produce library amplicons; and
d. assessing the presence of DNA damage based on the number of library amplicons produced.

Embodiment 49. The method of embodiment 48, wherein the forward primer and/or the reverse primer bind to one or more sequences comprised in one or both hairpin adapter.

Embodiment 50. The method of embodiment 48 or embodiment 49, wherein the forward primer binds to a sequence comprised in the hairpin adapter attached to a first end of the double-stranded DNA insert and the reverse primer binds to a sequence comprised in the hairpin adapter attached to a second end of the double-stranded DNA insert.

Embodiment 51. The method of any one of embodiments 48-50, wherein the number of library amplicons produced is estimated by measuring a cycle of quantification (Cq) value.

Embodiment 52. The method of any one of embodiments 48-51, wherein a higher number of library amplicons results in a lower Cq value.

Embodiment 53. The method of any one of embodiments 48-52, wherein a library with a lower Cq value has less DNA damage.

Embodiment 54. The method of any one of embodiments 51-53, further comprising determining conditions for analysis of the library based on the Cq value.

Embodiment 55. The method of embodiment 54, wherein the analysis is sequencing.

Embodiment 56. The method of any one of embodiments 48-55, wherein the amplifying is optimized for amplifying library molecules that are 5 kb or greater, 10 kb or greater, 15 kb or greater, 20 kb or greater, 25 kb or greater, or 30 kb or greater.

Embodiment 57. The method of any one of embodiments 48-56, wherein the amplifying is performed with a polymerase optimized for amplification of long amplicons.

Embodiment 58. The method of embodiment 57, wherein the polymerase is optimized for amplification of amplicons of 20 kb or more or 30 kb or more.

Embodiment 59. The method of embodiment 57 or embodiment 58, wherein the polymerase has a higher processivity or extension rate as compared to a wildtype Taq polymerase.

Embodiment 60. The method of embodiment 59, wherein the polymerase comprises one or more mutation or fusion that increase processivity or extension rate.

Embodiment 61. The method of embodiment 59 or embodiment 60, wherein the polymerase has an extension rate of greater than 3 kb/minute.

Embodiment 62. The method of any one of embodiments 48-61, wherein the amplifying is exponential.

Embodiment 63. The method of any one of embodiments 48-62, wherein or more or 40 or more cycles of amplifying are performed.

Embodiment 64. The method of any one of embodiments 48-63, wherein the DNA damage comprises one or more nicks in a library molecule.

Embodiment 65. The method of embodiment 64, wherein the one or more nicks are within the insert.

Embodiment 66. The method of embodiment 64 or embodiment 65, wherein the Cq value is greater when a greater percentage of library molecules in the library comprise one or more nicks.

Embodiment 67. The method of any one of embodiments 64-66, wherein the DNA damage comprises two or more nicks in a library molecule, wherein the nicks are in the same strand of the double-stranded DNA insert.

Embodiment 68. The method of any one of embodiments 64-66, wherein the DNA damage comprises two or more nicks in a library molecule, wherein the nicks are in both strands of the double-stranded DNA insert.

Embodiment 69. The method of any one of embodiments 48-68, wherein the forward primer and/or the reverse primer cannot generate an amplicon corresponding to the full sequence of the library molecule if the library molecule comprises one or more nicks.

Embodiment 70. The method of embodiment 69, wherein an amplicon generated from a library molecule comprising a nick lacks a sequence for binding to the forward and/or reverse primer.

Embodiment 71. The method of any one of embodiments 64-70, wherein library molecules comprising a nick generate fewer amplicons during the amplifying as compared to library molecules not comprising a nick.

Embodiment 72. The method of any one of embodiments 64-71, further comprising generating a double-stranded break from a nick before annealing the forward primer and the reverse primer.

Embodiment 73. The method of embodiment 72, wherein the generating a double-stranded break is performed using an enzymatic reaction.

Embodiment 74. The method of embodiment 73, wherein the enzymatic reaction is performed by an endonuclease.

Embodiment 75. The method of embodiment 74, wherein the endonuclease is a T7 endonuclease.

Embodiment 76. The method of any one of embodiments 72-75, wherein a library molecule comprising a double-stranded break does not generate amplicons corresponding to the full sequence of the library molecule during the amplifying.

Embodiment 77. The method of embodiment 72-76, wherein an amplicon generated from a library molecule comprising a double-stranded break lacks a sequence for binding to the forward and/or reverse primer.

Embodiment 78. A method of quantifying DNA damage in a sample comprising DNA using fluorescence comprising:
a. combining:
   i. an aliquot of a sample comprising DNA,
   ii. one or more DNA repair enzyme; and
   iii. dNTPs, wherein one or more dNTP is fluorescently labeled;
b. preparing repaired DNA;
c. dephosphorylating the phosphates from dNTPs;
d. binding the repaired DNA to carboxylate or cellulose beads;
e. eluting the bound repaired DNA from the carboxylate or cellulose beads with a resuspension buffer; and
f. measuring fluorescence of the repaired DNA to determine the amount of DNA damage.

Embodiment 79. The method of embodiment 78, wherein a greater fluorescence of the repaired DNA indicates greater DNA damage.

Embodiment 80. The method of embodiment 78 or embodiment 79, wherein the fluorescence of the repaired DNA is linear over a range of different amounts of DNA damage.

Embodiment 81. The method of any one of embodiments 78-80, wherein the assay can assess DNA damage induced by a manipulation of the sample by assessing an aliquot of the same sample before and after the manipulation.

Embodiment 82. The method of embodiment 81, wherein the manipulation is sequencing of a sample.

Embodiment 83. The method of embodiment 81 or embodiment 82, wherein measuring fluorescence of the repaired DNA comprises preparing a standard curve of dilutions of repaired DNA and measuring the fluorescence of the dilutions of repaired DNA.

Embodiment 84. The method of any one of embodiments 78-83, wherein measuring fluorescence of the repaired DNA comprises comparing the fluorescence of the repaired DNA against a separate standard curve of dilutions of only the one or more dNTP that is fluorescently labeled to determine the number of fluorescent dye molecules comprised in the repaired DNA.

Embodiment 85. The method of embodiment 84, further comprising calculating the normalized number of fluorescent dye molecules comprised in the repaired DNA by dividing the number of fluorescent dye molecules determined by the mass of the repaired DNA.

Embodiment 86. The method of any one of embodiments 78-85, wherein the DNA is genomic DNA, cDNA, or a library comprising fragmented double-stranded DNA.

Embodiment 87. The method of embodiment 86, wherein the DNA is genomic DNA and cDNA and the method further comprising preparing a library after determining the amount of DNA damage.

Embodiment 88. The method of embodiment 87, wherein a library is prepared if the amount of DNA damage is 5% or less, 4% or less, 3% or less, 2% or less, or 1% or less of total nucleotides.

Embodiment 89. The method of any one of embodiments 78-88, wherein a library is not prepared if the amount of DNA damage is 5% or greater, 4% or greater, 3% or greater, 2% or greater, or 1% or greater of total nucleotides.

Embodiment 90. The method of any one of embodiments 78-89, wherein more than one round of binding the repaired DNA to carboxylate or cellulose beads and eluting is performed before measuring the fluorescence.

Embodiment 91. The method of embodiment 90, wherein two rounds of binding the repaired DNA to carboxylate or cellulose beads and eluting is performed before measuring the fluorescence.

Embodiment 92. The method of any one of embodiments 78-91, wherein the carboxylate or cellulose beads are magnetic.

Embodiment 93. The method of any one of embodiments 78-92, wherein the preparing repaired DNA is performed at 37° C.

Embodiment 94. The method of any one of embodiments 78-93, wherein the preparing repaired DNA is performed for 10 minutes or more, 20 minutes or more, 30 minutes or more, 45 minutes or more, or 60 minutes or more.

Embodiment 95. The method of embodiment 78-94, wherein dephosphorylating the phosphates from dNTPs is performed with an enzyme.

Embodiment 96. The method of embodiment 78-95, wherein the enzyme for dephosphorylating the phosphates from dNTPs is shrimp alkaline phosphatase (SAP) or calf intestinal alkaline phosphatase (CIP).

Embodiment 97. The method of any one of embodiments 78-96, wherein the one or more DNA repair enzyme comprises a DNA polymerase.

Embodiment 98. The method of embodiment 97, wherein the DNA polymerase has 5′-3′ polymerase activity but lacks 5′-3′ exonuclease activity.

Embodiment 99. The method of embodiment 97, wherein the DNA polymerase is Bst DNA polymerase, large fragment.

Embodiment 100. The method of any of embodiments 78-99, wherein the one or more DNA repair enzyme comprises a ligase.

Embodiment 101. The method of embodiment 100, wherein the ligase is Taq ligase.

Embodiment 102. The method of any one of embodiments 78-101, wherein the DNA damage comprises a nick in double-stranded DNA.

Embodiment 103. The method of any one of embodiments 78-102, wherein the one or more DNA repair enzyme comprises T4 pyrimidine dimer glycosylase (PDG).

Embodiment 104. The method of any one of embodiments 78-103, wherein the DNA damage comprises a thymine dimer.

Embodiment 105. The method of embodiment 104, wherein the thymine dimer was induced by ultraviolet irradiation.

Embodiment 106. The method of any of embodiments 78-105, wherein the one or more DNA repair enzyme comprises uracil DNA glycosylase (UDG) and an apurinic or apyrimidinic site lyase.

Embodiment 107. The method of any one of embodiments 78-106, wherein the DNA damage comprises a uracil.

Embodiment 108. The method of any of embodiments 78-107, wherein the one or more DNA repair enzyme comprises formamidopyrimidine DNA glycosylase (FPG) and an apurinic or apyrimidinic site lyase.

Embodiment 109. The method of embodiment 78-108, wherein the DNA damage comprises an oxidized base.

Embodiment 110. The method of any one of embodiments 78-109, wherein the dNTPs comprise dATP, dGTP, dCTP, and dTTP or dUTP.

Embodiment 111. The method of any one of embodiments 78-110, wherein all the dNTPs are fluorescently labeled.

Embodiment 112. The method of embodiment 78-111, wherein dUTP and dCTP are fluorescently labeled.

Embodiment 113. The method of embodiment 112, wherein the fluorescent label is Alexa Fluor 488, Alexa Fluor 546, Alexa Fluor 555, Alexa Fluor 633, fluorescein isothiocyanate (FITC), or tetramethylrhodamine-5-(and 6)-isothiocyanate (TRITC).

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows a representative standard method for large indel detection. Such methods involve low-cycle PCR around a cut site (low cycles, ˜1 kb wildtype amplicon) with the PCR conditions optimized for long amplicons (˜10 kb). After amplification, Nextera library prep (LP) is performed on PCR amplicons. Amplicon analysis involves “de novo” amplicon assembly and quantification of unique gene-editing events (i.e., events that generate unique amplicons).

FIGS. 2A and 2B summarize long amplification (LongAmp) insertion controls that can be prepared using a universal UMI double-stranded (ds) DNA oligonucleotide. The UMI dsDNA oligonucleotide can be commercially sourced (such as a gBlock gene fragment from Integrated DNA Technologies) (A). This oligonucleotide can be used to prepare LongAmp insertion controls (B). RS (in RS1, etc.) refers to a restriction site. N18 refers to a UMI sequence comprising 18 random nucleotides. LA-fwd and LA-rev refer, respectively, to forward and reverse primers for the LongAmp reactions. Controls 1, 2, 3, and n comprise inserts of 0.2 kb, 1 kb, 2 kb, and 10 kb, respectively. The bright region of the 10 kb standard indicates that this standard is not drawn to scale.

FIG. 3 shows a method of producing an upstream universal PCR adapter amplicon and a downstream universal PCR adapter amplicon. These amplicons may be used as a 5′ universal oligonucleotide and a 3′ universal oligonucleotide, respectively. Primers comprising RS1 and RS2 and that bind on complementary strands in a 5′ region or 3′ region in a target sequence of interest can be used to generate an upstream universal PCR adapter amplicon (5′ region) and a downstream universal PCR adapter amplicon (3′ region) using the LA-amp forward and reverse primers, respectively (for example, with the LA-fwd/RS1 primers for upstream amplicons and LA-rev/RS2 for downstream amplicons). The “cut site” shown refers to a cut site introduced via gene editing (such as with a CRISPR Cas system) into a representative sequence of interest, as insertion and deletions may often occur around such cut sites used for gene editing. Other sequences of interest (such as those comprised in samples from cancer patients being evaluated for insertion/deletion mutations) would not have an introduced cut site.

FIG. 4 shows a method of preparing insertion amplicons of different sizes using tailed PCR primers. The method uses a set of two primers that comprise sequences of restriction enzyme cleavage sites (RS's) and that bind to primer binding sequences within a sequence of interest (i.e., two primers such as those comprising RS1/RS3 sequences or two primers such as those comprising RS2/RS4 as shown). The sizes of insertion amplicons and insertion amplicons can be controlled by the choice of primers based on their primer binding sites with the sequence of interest. In this figure, upstream refers to a sequence in a 5′ portion of the sequence of interest and downstream refers to a sequence in a 3′ portion of the sequence of interest. An insertion amplicon pair can refer to an upstream insertion amplicon and a downstream insertion amplicon. The bright region of the 10 kb standard indicates that this standard is not drawn to scale.

FIG. 5 shows a method of producing deletion standards. Primers that bind RS3 and RS4 on complementary strands of the sequence of interest can be used to generate deletion amplicons using the LA-amp forward and LA-amp reverse primers (for example, with the LA-fwd/RS3 primers or LA-rev/RS4). A deletion amplicon pair can refer to an upstream deletion amplicon and a downstream deletion amplicon. The restriction sites corresponding to RS3 and RS4 can then be used to generate proper ends for ligating the cut amplicons to universal UMI ds DNA oligonucleotides (as shown in FIG. 6A) to generate LongAmp deletion standards as shown in FIG. 6B.

FIGS. 6A and 6B summarize long amplification (LongAmp) deletion controls that can be prepared using a universal UMI double-stranded (ds) DNA oligonucleotide. The UMI dsDNA oligonucleotide can be commercially sourced (such as a gBlock gene fragment from Integrated DNA Technologies) (A). This oligonucleotide can be used to prepare LongAmp deletion standards (B). Controls 1, 2, 3, and n comprise deletions of −20 base pairs (bp), −50 bp, or approximately −1 kb, respectively.

FIG. 7 shows the mass of control inputs that may be in a LongAmp reaction to avoid duplicates of UMI sequences.

FIGS. 8A-8C shows representative individual standards that may be comprised in a pool of nucleic acid standards of different lengths. These standards may all comprise a UMI, as well as LA-rev and LA-fwd primer binding sequences. Table 1 below provides descriptors for the labeled regions and oligonucleotides comprised in the standards. A full-length standard may comprise a 5′ universal oligonucleotide and a 3′ universal oligonucleotide (100 and 101) (A). An insertion standard may comprise a 5′ universal oligonucleotide, a 3′ universal oligonucleotide, and a region between a UMI and a 5′ universal oligonucleotide and a region between the UMI and a 3′ universal oligonucleotide (100, 101, and 102 and 103) (B). An insertion standard may also comprise either a region between a UMI and a 5′ universal oligonucleotide or a region between the UMI and a 3′ universal oligonucleotide, but not both regions (as shown in bottom standard of 8B comprising 100, 101, and 103, but not 102). A deletion standard may comprise a 5′ partially overlapping oligonucleotide and a 3′ partially overlapping oligonucleotide (104 and 105) (C). A deletion standard may comprise either a 5′ partially overlapping oligonucleotide or a 3′ partially overlapping oligonucleotide, but not both (as shown in bottom standard of 8C comprising 104, but not 105). As described herein, a pool of nucleic acid standards may comprise any or all the different types of standards shown here.

TABLE 1
Description of labels

| Label | Description |
|---|---|
| 100 | 5' universal oligonucleotide |
| 101 | 3' universal oligonucleotide |
| 102 | region between a UMI and a 5' universal oligonucleotide |
| 103 | region between a UMI and a 3' universal oligonucleotide |
| 104 | 5' partially overlapping oligonucleotide |
| 105 | 3' partially overlapping oligonucleotide |

FIG. 9 summarizes a quantitative PCR (qPCR) assay for assessing DNA damage in long libraries. The assay uses forward and reverse primers that bind to sequences within hairpin adapters comprised in library molecules. Libraries without DNA damage (such as nicks) will generate more signal (i.e. produce more full-length amplicons). As shown in the figure, an exemplary assay may include exponential amplification with a polymerase optimized for LongAmp PCR (such as PrimeStar GXL DNA polymerase, Takara).

FIGS. 10A-10D show results of average cycle of quantification (Cq) and % damage with the QC assay for libraries treated with different concentrations of nickase. Cq (A) and % damage (B) results are shown for a 10 ng library, as well as Cq (C) and % damage (D) results for a 20 ng library.

FIG. 11 shows results from a method of converting nicks in library molecules into double-stranded breaks, such as with a combination of Vibrio vulnificus nuclease (VVN) and a T7 endonuclease mutant. Endo=endonuclease.

FIGS. 12A and 12B summarize how Cq values differ when library is treated or not treated with an endonuclease mutant. (A) Summary of Cq values. (B) Summary of automated electrophoresis results using TapeStation®, Agilent.

FIGS. 13A-13C show results when SMRTbell templates were assessed in quantitative PCR (qPCR) and then sequenced on the PacBio Sequel 2 system to determine whether qPCR Cq's correlate with sequencing metrics. Samples are ordered from lowest to highest Cq. (A) Average Cq. (B) Total ouput. (C) Variation (% P1). Correlation is observed for qPCR Cq and total output (gigabases, GB), and a lower the Cq indicates a higher output (with the exception of one outlier of Library 8, the lowest Cq). Generally, the libraries had an average Cq value of 2-3. The qPCR results predicted Library 13 to be low in quality, which is confirmed by relatively poor sequencing results.

FIGS. 14A-14C show data with another set of SMRTbell templates assessed in qPCR and then sequenced on the PacBio Sequel 2 system. (A) Average Cq values, with samples ordered from lowest to highest Cq. (B) Total output (GB). (C) Percentage P1. Correlation is observed for qPCR Cq and total output, and a lower Cq indicates a higher output (with the exception of one outlier of Library 14, the lowest Cq). Most libraries had an average Cq value of 3-4. The qPCR predicted Library 10 to be low in quality, which is confirmed by sequencing.

FIGS. 15A-15C show data on the qPCR QC assay results for several PacBio SMRTbell libraries pre-sequencing and correlated to total Gb output. Total output increases with lower Cq values suggesting this QC assay could serve as a useful tool to predict sequencing performance. Cq values and Gb measurements for library fractions (F#) from Library 20 (A), Library 21 (B), and Library 22 (C).

FIG. 16 shows a DNA damage detection workflow. The signal-to-noise ratio of this assay was increased by employing both a shrimp alkaline phosphatase (SAP) digestion and a stringent double-SPRI bead-based purification step (i.e., two purifications with carboxylate beads) to greatly reduce nonspecific binding of unincorporated fluorescent nucleotides.

FIG. 17 shows results of SAP digestion and a single SPRI bead-based purification step. Single SPRI-purified sheared and genomic DNA demonstrated reduced nonspecific binding of fluorescent nucleotides when treated with SAP before purification (+SAP) as opposed to without SAP treatment (−SAP).

FIG. 18 shows that two bead-based purification steps substantially reduced nonspecific binding of fluorescent nucleotides.

FIGS. 19A and 19B show a comparison of the efficacy of a commercially available repair mix (PreCR Repair mix (NEB), shown in panel (A)) and the present method with a DNA repair enzyme mix comprising Taq ligase (40 U), Bst polymerase large fragment (8 U), and T4 PDG (1 U) (shown in panel (B)).

FIG. 20 shows measurement of ultraviolet (UV) damage to genomic DNA samples. As the energy of the light increases and the exposure time increases, the amount of fluorescence also increases in samples repaired with a custom DNA repair enzyme mix comprising Taq ligase, Bst polymerase, and T4 pyrimidine dimer glycosylase (T4 PDG), a UV-damage specific repair enzyme.

FIG. 21 shows measurement of nicking damage to genomic DNA samples. As the amount of nicking enzyme (Nt.BspQI) increases, the fluorescence signal generally also increases in samples repaired with Taq ligase and Bst polymerase using the present assay.

### DESCRIPTION OF THE EMBODIMENTS

Long amplification PCR can be used for targeted long indel detection in a sequence of interest from a target nucleic acid. However, PCR is biased towards smaller amplicons, such as those with small insertions and deletion mutations, and biased against longer amplicons, such as long insertions. This bias is inherent in PCR methods, as longer amplicons will take longer for synthesis of a new strand of nucleic acid with a lower likelihood that a longer amplicon is produced over a PCR cycle, as compared to shorter amplicons. Further, longer amplicons will have a lower rate of success in producing the full amplicon before an event may stop replication. In other words, amplification of longer amplicons may fail with a higher rate than that of shorter amplicons. For example, the longer a polymerase must work to produce an amplicon, the greater the chance it will not reach the end of an amplicon due to random falling off, encountering DNA damage, or lack of time given its rate of processivity.

Because of the known bias against long amplicons, long amplification (LongAmp) PCR cannot be used to accurately determine the relative frequency of different events. Thus, the results of LongAmp amplification cannot quantify the relative number of specific mutations in the original target nucleic acid sample, because the size of the amplicons associated with different mutations will amplify differently.

The standards and methods described herein can help to normalize for this amplicon size bias.

Further, this disclosure also describes a quality control (QC) method for assessing library quality. In some embodiments, a library, such as one for long-read sequencing, is assessed prior to sequencing. In some embodiments, a library comprises library molecules comprising double-stranded DNA inserts with a hairpin adapter at both ends of the inserts. In some embodiments, the library is generated by fragmenting target DNA and incorporating hairpin adapters at both ends of fragments, such as with tagmentation or ligation.

### I. Standards for Normalizing Amplicon Size Bias

In some embodiments, a pool of nucleic acid standards of different lengths can be used in methods to normalize for amplicon size bias. In some embodiments, these nucleic acid standards comprise a unique molecular identifier (UMI).

In some embodiments, a pool of nucleic acids may comprise a range of different sequences comprised in a sequence of interest.

In some embodiments, the number of standards in the pool is greater than the number of amplicons generated by an amplification reaction. In some embodiments, the amplification reaction is amplification of a sequence of interest.

In some embodiments, at least a first portion of the standards are from one pool of standards and wherein at least a second portion of the standards are from another pool of standards.

In some embodiments, the standards are double-stranded. In some embodiments, the standards comprise double-stranded DNA. In some embodiments, each standard comprises a different UMI.

In some embodiments, an amplification primer binding sequence is comprised at or in close proximity to one or both ends of each standard. Throughout this document, “in close proximity to one or both ends” means within 10 or fewer nucleotides of the end. In some embodiments, an amplification primer binding sequence is comprised at the end of one or both ends of each standard. In some embodiments, an amplification primer binding sequence is comprised with 1, 2, 3, 4, 5, 6, 7, 8, or 9 nucleotides of one or both ends of each standard. In some embodiments, a standard comprises an amplification primer binding sequence at both its 3′ end and its 5′ end. In some embodiments, a standard comprises a different amplification primer binding sequence at 3′ end versus its 3′ end. In some embodiments, a standard comprises one or more oligonucleotide 5′ of the UMI. In some embodiments, a standard comprises one or more oligonucleotide 3′ of the UMI. In some embodiments, a standard comprises one or more oligonucleotide 5′ of the UMI and one or more oligonucleotide 3′ of the UMI.

A. UMIs

In some embodiments, the standards in the pool of standards each comprise a UMI.

In some embodiments, a UMI is not at or in close proximity to the 5′ and/or 3′ end a standard. In some embodiments, a UMI that is located centrally within a standard increases the probability that fragmentation of the standard (such as by tagmentation) yields fragments comprising the UMI and all or part of a sequence from the rest of the standard (either 5′ and/or 3′ of the UMI). As used herein, a “centrally” located feature refers to the middle of the feature being at a position within 10 or fewer nucleotides of the center of a standard. In some embodiments, a UMI located centrally within a standard has the middle of the UMI within 1, 2, 3, 4, 5, 6, 7, 8, or 9 nucleotides of the center of the standard.

Placing the UMI proximal to the 5′ and/or 3′ end of the sequence of interest, in contrast, might lead to a higher percentage of fragments that comprise only the UMI and not additional sequence from the rest of the standard.

In some embodiments, UMIs are used to identify amplicons that are generated from the same LongAmp standard. In other words, sequencing of standards comprising a UMI and upstream/downstream insertion junction bases can provide the unique molecule count and control identity of the standard, respectively. This is because each amplicon generated from the same standard will have the same unique UMI, and other amplicons generated from LongAmp standards will have different UMIs.

In some embodiments, the UMIs comprises random base pairs, such that each unique UMI comprises a different sequence from other UMIs in the pool. In some embodiments, the UMI comprises 10 (N10) or more, 12 (N12) or more, 14 (N14) or more, 16 (N16) or more, 18 (N18) or more, 20 (N20) or more, or 22 (N22) or more random base pairs. In some embodiments, the UMI comprises 18 base pairs (N18). In some embodiments, the UMIs comprised in the pool of standards are a random set of sequences comprising 16-20 base pairs.

Use of a UMI pool having a large number of UMIs (can help to avoid UMI collision. Having a longer UMI (i.e., N18 instead of N10) also reduces the chances of UMI collision.

As used herein, “UMI collision” refers to the event of observing two reads with the same sequence and same UMI barcode but originating from two different genomic molecules. With amplicon sequencing, a specific location in the genome is sequenced many times, resulting in sequencing depth much greater than genome-wide sequencing (See Clement et al., Bioinformatics, 34, 2018, i202-i210). Based on this sequencing depth, many alleles from different genomic molecules may share the same sequence, and the possibility of UMI collisions is much higher for amplicon sequencing compared with whole genome sequencing.

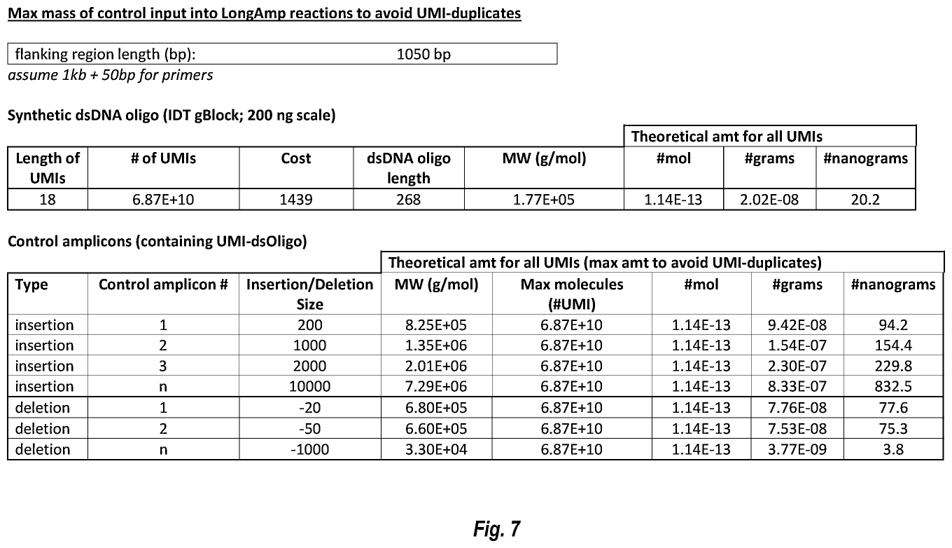

In some embodiments, the pool of standards comprises 1×10¹⁰ or greater, 10×10¹⁰ or greater, or 100×10¹⁰ or greater standards, wherein each standard comprises a different UMI. FIG. 7 shows calculations for preparing an experiment comprising 6.87×10¹⁰ UMIs, including an amount of synthetic double-stranded DNA comprising UMIs needed.

In some embodiments, UMIs in standards may originate from relatively inexpensive commercially available reagents, as described herein. In some embodiments, a double-stranded oligonucleotide comprising a UMI also comprises one or more restriction enzyme cleavage sites for use in preparing standards.

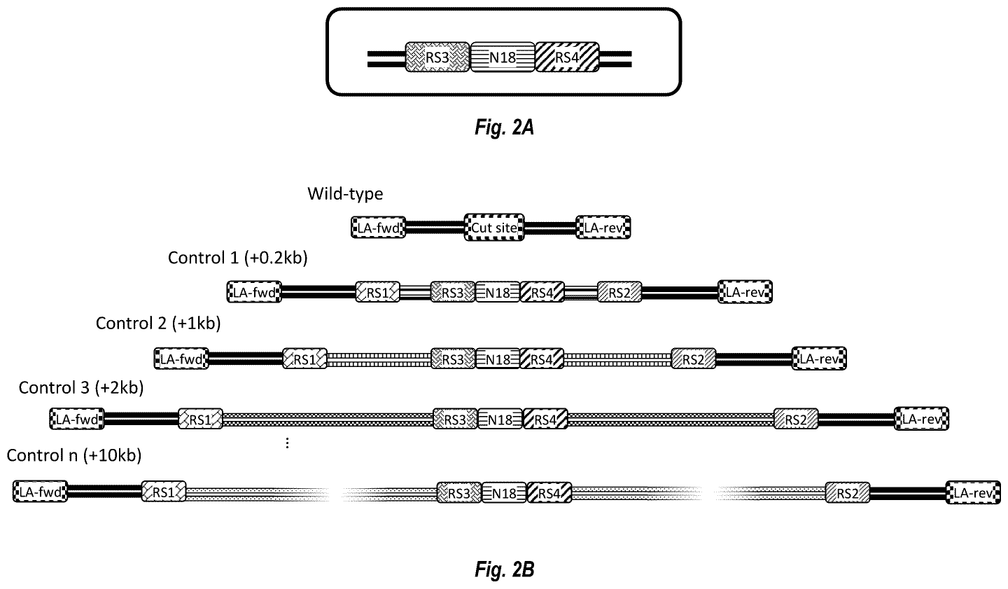

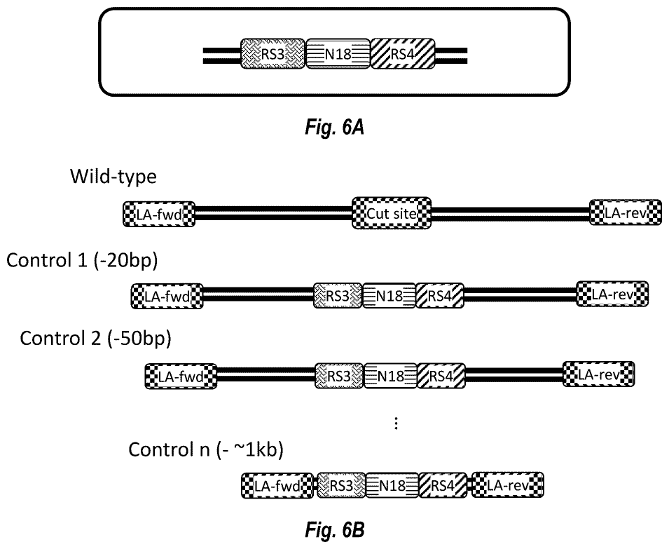

For example, representative synthetic dsDNA oligonucleotides are shown for preparing insertion standards (FIG. 2A) and for preparing deletion standards (FIG. 6A), as described below. In some embodiments, a synthetic dsDNA oligonucleotide comprises a UMI and restriction enzyme cleavage sites (or restriction sites, such as RS3 and RS4, as shown in FIGS. 2A and 6A). In some embodiments, the restriction enzyme cleavage sites can be used to cut the oligonucleotide and then ligate to other oligonucleotides to prepare the final standards. Sources of UMI dsDNA oligonucleotides include gBlock gene fragments (Integrated DNA Technologies).

B. Sequence of Interest

As used herein a “sequence of interest” can be any sequence that a user wants to investigate. In some embodiments, the sequence of interest has been subjected to gene editing. For example, a user may have performed a method of gene editing or other mutagenesis (such as chemical mutagenesis) and wants to evaluate the different mutations (along with the wild-type sequence) in the sequence of interest.

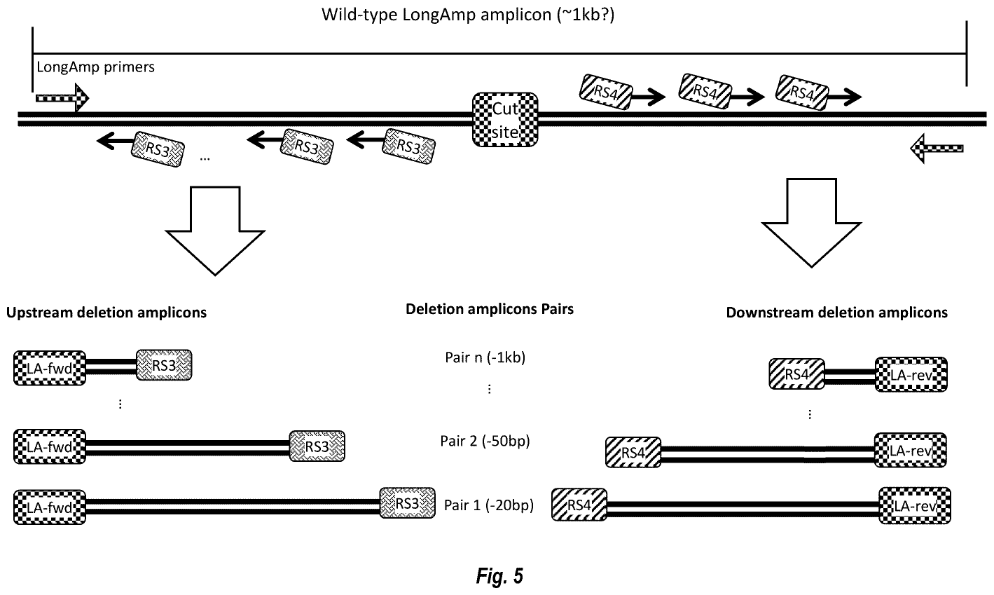

In some embodiments, the gene editing is performed with a CRISPR Cas method. In some embodiments, a CRISPR Cas cut site is present in the sequence of interest. In some embodiments, insertion or deletion mutations are likely to occur near a cut site within a sequence of interest. For example, FIG. 5 shows a cut site present within a sequence of interest that has been introduced using a method of gene editing, such as CRISPR Cas. Some sequences of interest, such as sequences from oncology samples from a patient that are being evaluated for indel mutations, would not have cut sites introduced by a gene editing methodology.

In some embodiments, the sequence of interest comprises a restriction enzyme cleavage site that is not at or in close proximity to the 5′ and/or 3′ end of the sequence of interest. In some embodiments, such a cut site may be of use in generating standards or may be used to evaluate the sequence of interest.

In some embodiments, the sequence of interest comprises a primer binding sequence capable of binding to long amplification primers (i.e., the LA-fwd and LA-rev primers). In some embodiments, a user can evaluate the sequence of interest to prepare appropriate LA-fwd and LA-rev primers.

In some embodiments, the sequence of interest may comprise insertion or deletion mutations. For example, the sequence of interest may comprise insertion mutation or may be a deletion mutation (i.e., not comprise the full sequence of the sequence of interest).

As used herein, the “wild-type” sequence of interest refers to a sequence of interest that does not comprise an indel mutation. In other words, the wild-type sequence refers to a sequence that does not comprise an insertion mutation and also does not comprise a deletion mutation. As used herein, a “wild-type amplicon” is an amplicon that comprises the wild-type sequence of interest.

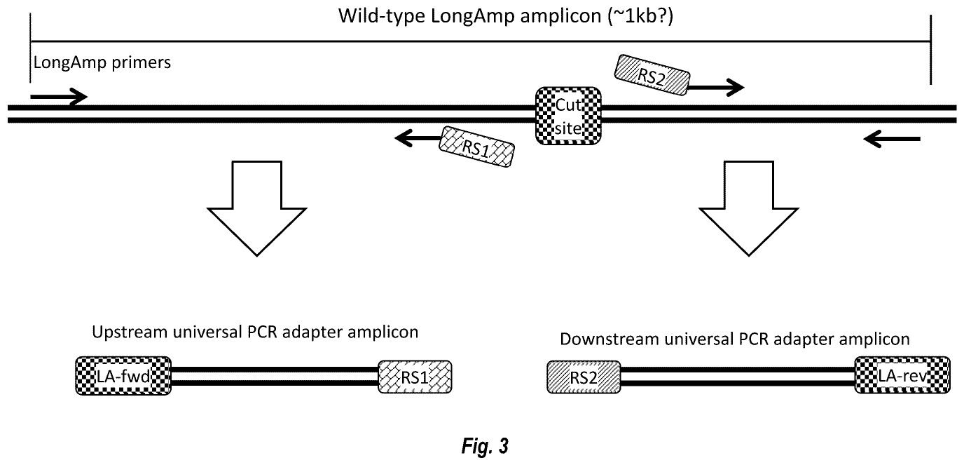

The sequence of interest can be any type of nucleic acid sequence. In some embodiments, the sequence of interest has been subject to gene-editing methods (such as CRISPR), and the user wants to analyze unique gene-editing events. In some embodiments, a sequence of interest that has been subjected to gene-editing may comprise a “cut site” as shown in representative examples in FIGS. 3, 5, and 6B. Such gene editing methods can lead to a variety of different types of indel mutations that a user may wish to characterize.

In some embodiments, sequences of interest comprising cancer and germline indel mutations could be evaluated by this method, as could insertions from transposable elements. In such embodiments, the sequence of interest may not comprise a cut site from a gene editing method.

In some embodiments, the sequence of interest may be all or part of a gene of interest, for example a gene known to be associated with cancer. One skilled in the art may want to characterize indels that a patient may have in a gene comprising a sequence of interest and/or characterize the relative amounts of different mutations. For example, one skilled in the art might want to characterize the number of large insertion mutations that are present in a sequence of interest from a patient's sample.

C. Standards Comprising a Universal Oligonucleotide

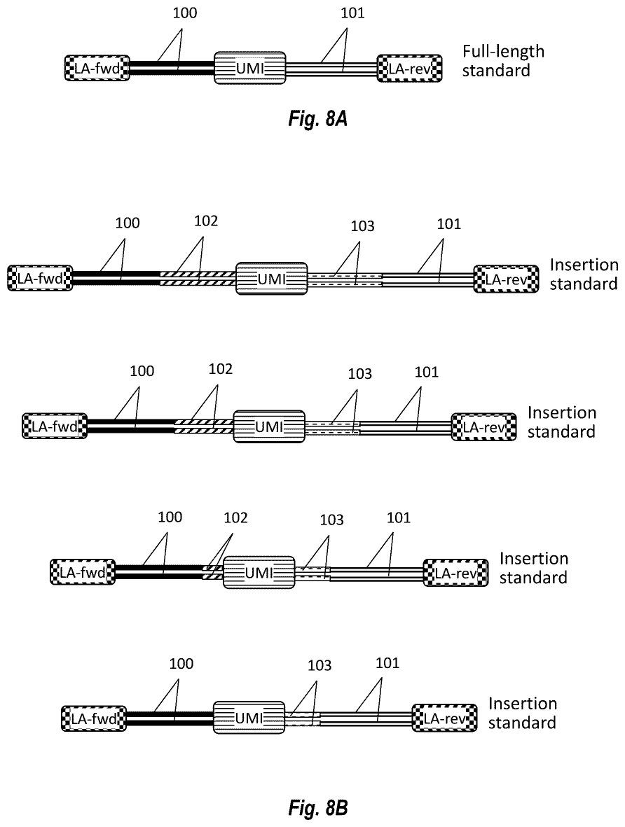

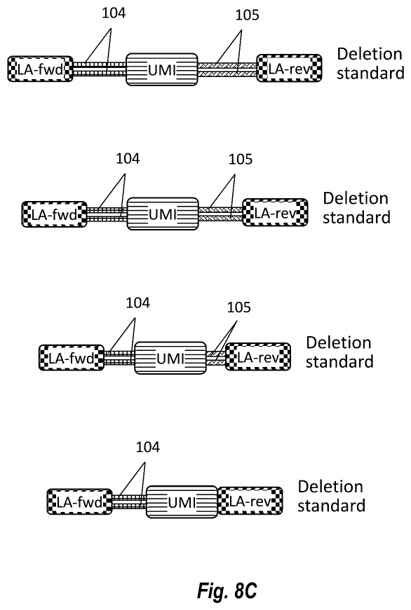

In some embodiments, all or some standards within a pool of nucleic acid standards comprise a 5′ universal oligonucleotide and a 3′ universal oligonucleotide. As used herein, a “universal oligonucleotide” refers to an oligonucleotide that is comprised in all the standards in this pool. As used herein, a “5′ universal oligonucleotide” is an oligonucleotide that is 5′ of a UMI comprised in the standard (as represented as 100 in FIG. 8). As used herein, a “3′ universal oligonucleotide” is an oligonucleotide that is 3′ of a UMI comprised in the standard (as represented by 101 in FIG. 8).

In some embodiments, at least a first portion of the standards are from one pool of standards and wherein at least a second portion of the standards are from another pool of standards. In other words, a pool of standards, wherein each standard comprises a 5′ universal oligonucleotide and a 3′ universal oligonucleotide, may be combined with a different pool of standards that do not comprise a 5′ universal oligonucleotide and/or a 3′ universal oligonucleotide.

In some embodiments, a pool of nucleic acid standards comprises standards of different lengths, wherein the nucleic acid standards comprise a unique molecular identifier (UMI) and a 5′ universal oligonucleotide, wherein the 5′ universal oligonucleotide is the same for all standards; a 3′ universal oligonucleotide, wherein the 3′ universal oligonucleotide is the same for all standards; and at least one region between the UMI and the 5′ universal oligonucleotide and/or between the UMI and the 3′ universal oligonucleotide; wherein the length of the at least one region determines the length of the standard. A region between the UMI and the 5′ universal oligonucleotide is shown as 102 in FIG. 8B, and a region between the UMI and the 3′ universal oligonucleotide is shown as 103 in FIG. 8B.

In some embodiments, a standard comprising a 5′ universal oligonucleotide and a 3′ universal oligonucleotide and also comprising additional sequence (such as a region between the UMI and the 5′ universal oligonucleotide and/or a region between the UMI and the 3′ universal oligonucleotide) may be referred to as an “insertion standard.” This is because an insertion standard may be longer in length that the wild-type sequence of interest. In this way, an insertion standard can control for normalizing amplicon size bias of insertion mutations in the wild-type sequence of interest, as these insertion mutations would be larger than the wild-type sequence of interest.

In some embodiments, the pool further comprises a nucleic acid standard that comprises a UMI and a 5′ universal oligonucleotide, wherein the 5′ universal oligonucleotide is the same for all standards; and a 3′ universal oligonucleotide, wherein the 3′ universal oligonucleotide is the same for all standards; wherein the further nucleic acid standard does not comprise at least one region between the UMI and the 5′ universal oligonucleotide or between the UMI and the 3′ universal oligonucleotide. A standard comprising a 5′ universal oligonucleotide (100) and a 3′ universal oligonucleotide (101), may be termed a full-length standard, as shown in FIG. 8A. A full-length standard may have a similar length as the wild-type sequence of interest without either an insertion or deletion mutation (i.e., the wild-type sequence without an indel).

In some embodiments, the at least one region between the UMI and the 5′ universal oligonucleotide and/or between the UMI and the 3′ universal oligonucleotide determines the length of an insertion standard. In some embodiments, the at least one region between the UMI and the 5′ universal oligonucleotide and/or between the UMI and the 3′ universal oligonucleotide comprise a number of kilobases (kb) that correspond to potential length of insertion mutations of interest. In some embodiments, the at least one region between the UMI and the 5′ universal oligonucleotide and/or between the UMI and the 3′ universal oligonucleotide comprise 0.2 kb-10 kb.

The 5′ universal oligonucleotide and/or the 3′ universal oligonucleotide may comprise a sequence comprised in the sequence of interest. In some embodiments, the 5′ universal oligonucleotide and/or the 3′ universal oligonucleotide each comprise an amplicon amplified from a sequence of interest. In other words, the 5′ universal oligonucleotide and/or the 3′ universal oligonucleotide may be prepared by amplification, as shown in FIG. 3.

When a 5′ universal oligonucleotide is prepared by amplification, it may be referred to as a “5′ universal PCR adapter amplicon” or “upstream universal PCR adapter amplicon.” FIG. 3 shows how representative upstream universal PCR adapter amplicons can be generated using the long amplification forward primer (LA-fwd) and a primer that binds to the sequence of interest and that comprises a restriction enzyme cleavage site (RS1).

When a 3′ universal oligonucleotide is prepared by amplification, it may be referred to as a “3′ universal PCR adapter amplicon” or “downstream universal PCR adapter amplicon.” FIG. 3 shows how representative downstream universal PCR adapter amplicons can be generated using the long amplification reverse primer (LA-rev) and a primer that binds to the sequence of interest and that comprises a restriction enzyme cleavage site (RS2).

In some embodiments, an upstream universal PCR adapter amplicon and a downstream universal PCR adapter amplicon may be cleaved with appropriate restriction enzymes (that can cleavage at RS1 and RS2 for the example shown in FIG. 3) to prepare standards comprising a UMI and a 5′ universal oligonucleotide, wherein the 5′ universal oligonucleotide is the same for all standards; and a 3′ universal oligonucleotide, wherein the 3′ universal oligonucleotide is the same for all standards. This cleavage may produce ends that are compatible for ligating these amplicons to other portions of the standards (such as a region between the UMI and the 5′ universal oligonucleotide and/or between the UMI and the 3′ universal oligonucleotide), as discussed below in the description of methods of making standards.

In some embodiments, the at least one region between the UMI and the 5′ universal oligonucleotide and/or between the UMI and the 3′ universal oligonucleotide each comprise an arbitrary sequence. As used herein, an “arbitrary sequence” refers to any sequence comprising nucleotides, without any requirement that a specific nucleic acid sequence is comprised in the arbitrary sequence. For example, one skilled in the art may want to prepare insertion standards wherein the arbitrary sequence is random and not related to the sequence of interest. In another embodiment, the arbitrary sequence may be a known sequence that is not random, but it is also not related to the sequence of interest (such as an unrelated gene sequence). Standards comprising an arbitrary sequence may be used to normalize for amplicon size bias of insertion mutations, as much of this bias is related to amplicon size and not to the exact sequence comprised in the inserted sequence. In some embodiments, the arbitrary sequence is double-stranded.

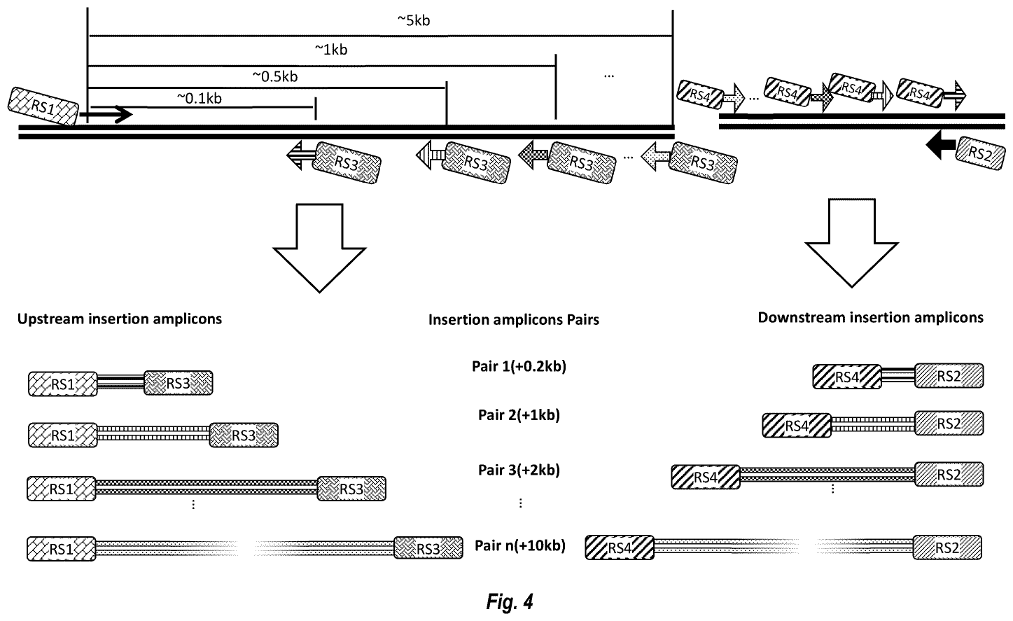

In some embodiments, the at least one region between the UMI and the 5′ universal oligonucleotide and/or between the UMI and the 3′ universal oligonucleotide each comprise an amplicon amplified from a sequence of interest. In other words, a region between the UMI and the 5′ universal oligonucleotide and/or between the UMI and the 3′ universal oligonucleotide may be prepared by amplification. In some embodiments, this amplification is from the sequence of interest, as shown in FIG. 4.

1. Insertion Amplicons

As used herein, a region between the UMI and the 5′ universal oligonucleotide, when prepared by amplification, may be referred to as a “5′ insertion amplicon” or an “upstream insertion amplicon.” FIG. 4 shows how representative upstream insertion amplicons can be generated using the primers that binds to the sequence of interest and that comprises a restriction enzyme cleavage sites (RS1 and RS3).

As used herein, a region between the UMI and the 3′ universal oligonucleotide, when prepared by amplification, may be referred to as a “3′ insertion amplicon” or an “downstream insertion amplicon.” FIG. 4 shows how representative upstream insertion amplicons can be generated using restriction enzyme cleavage sites (RS2 and RS4).

In some embodiments, the reverse and forward primers used for preparing insertion amplicons determines the size of the insertion amplicon. In some embodiments, a single primer pair generates an insertion amplicon of a desired size.

As used herein, “an insertion amplicon” can refer to an amplicon that is either a 5′ insertion amplicon or a 3′ insertion amplicon. Generally, “an insertion amplicon” is not limited by its placement in a standard.

In some embodiments, a standard comprises both an upstream insertion amplicon and a downstream insertion amplicon (as shown in FIG. 4). These may be referred to as “insertion amplicon pairs.” However, a standard may also only comprise either an upstream insertion amplicon or a downstream insertion amplicon.

FIG. 2B shows a representative pool of standards comprising a pool of nucleic acid standards comprise a 5′ universal oligonucleotide and a 3′ universal oligonucleotide. As shown in FIG. 2B, the pool of standards may comprise an upstream insertion amplicon and a downstream insertion amplicon, prepared as shown in FIG. 4.

D. Standards Comprising a Partially Overlapping Oligonucleotide

In some embodiments, a pool of nucleic acid standards of different lengths comprises nucleic acid standards comprising a UMI and a 5′ partially overlapping oligonucleotide, wherein the 5′ partially overlapping oligonucleotide is identical over at least a portion of its sequence for all the standards; and/or a 3′ partially overlapping oligonucleotide, wherein the 3′ partially overlapping oligonucleotide is identical over at least a portion of its sequence for all the standards; wherein the lengths of the 5′ partially overlapping oligonucleotide and/or the 3′ partially overlapping oligonucleotide determines the length of the standard.

As used herein, a “partially overlapping oligonucleotide” refers to an oligonucleotide that is identical over at least a portion of its sequence for all the standards. In some embodiments, a standard comprises both a 5′ partially overlapping oligonucleotide and a 3′ partially overlapping oligonucleotide.

As used herein, a “5′ partially overlapping oligonucleotide” is an oligonucleotide that is 5′ of a UMI comprised in the standard, as represented by 104 in FIG. 8C. As used herein, a “3′ partially overlapping oligonucleotide” is an oligonucleotide that is 3′ of a UMI comprised in the standard, as represented by 105 in FIG. 8C. In some embodiments, the 5′ partially overlapping oligonucleotide and the 3′ partially overlapping oligonucleotide are different. In some embodiments, the 5′ partially overlapping oligonucleotide and the 3′ partially overlapping oligonucleotide comprise different numbers of nucleotides.

In some embodiments, the 5′ partially overlapping oligonucleotide comprises at least a first portion of a sequence of interest and the 3′ partially overlapping oligonucleotide comprise at least a second portion of a sequence of interest. In other words, the 5′ partially overlapping oligonucleotide comprises at least a first portion of a sequence of interest and the 3′ partially overlapping oligonucleotide may correspond to different portions of a sequence of interest.

In some embodiments, a standard only comprises a 5′ partially overlapping oligonucleotide (and not a 3′ partially overlapping oligonucleotide). In some embodiments, a standard only comprises a 3′ partially overlapping oligonucleotide (and not a 5′ partially overlapping oligonucleotide). A standard that comprises only a 5′ partially overlapping oligonucleotide or a 3′ partially overlapping oligonucleotide may be useful to control for a deletion mutation that results in a loss of a large region in a sequence of interest.

In some embodiments, the 5′ partially overlapping oligonucleotide and/or the 3′ partially overlapping oligonucleotide each comprise an amplicon amplified from a sequence of interest, as shown in FIG. 5.

1. Deletion Amplicons

A 5′ partially overlapping oligonucleotide, when generated by amplification from a sequence of interest, may be termed a 5′ deletion amplicon or an upstream deletion amplicon. A 3′ partially overlapping oligonucleotide, when generated by amplification from a sequence of interest, may be termed 3′ deletion amplicon or a downstream deletion amplicon. For example, as shown in FIG. 5, each of the upstream deletion amplicons comprises a portion of the sequence of interest (shown in black) and each of the downstream deletion amplicons also comprises a portion of the sequence of interest (shown in black). In some embodiments, the portion of the sequence of interest comprised in the upstream deletion amplicons and downstream deletion amplicons may be different. FIG. 5 shows how representative upstream deletion amplicons and downstream deletion amplicons can be generated using the primers that comprises a restriction enzyme cleavage sites (such as RS3 and RS4) and that bind to the LA-fwd and LA-rev primer binding sequences and other sequences comprised in the sequence of interest.

As used herein, “a deletion amplicon” can refer to an amplicon that is either a 5′ deletion amplicon or a 3′ deletion amplicon. Generally, “a deletion amplicon” is not limited by its placement in a standard.

In some embodiments, the reverse and forward primers used for preparing a deletion amplicon determines the size of the deletion amplicon. In some embodiments, a single primer pair generates a deletion amplicon of a desired size.

In some embodiments, a standard comprises both an upstream deletion amplicon and a downstream deletion amplicon (as shown in FIG. 5). These may be referred to as “deletion amplicon pairs.” However, a standard may also only comprise either an upstream deletion amplicon or a downstream deletion amplicon.

In some embodiments, the 5′ partially overlapping oligonucleotide and/or the 3′ partially overlapping oligonucleotide each comprise a sequence that is 20 bp-1 kb smaller than a sequence of interest. In other words, 5′ partially overlapping oligonucleotide and/or the 3′ partially overlapping oligonucleotide may correspond to a sequence found in a deletion mutation of the sequence of interest.

FIG. 6B shows a representative pool of standards comprising a pool of nucleic acid standards comprising an upstream deletion amplicon and a downstream deletion amplicon, prepared as shown in FIG. 5.

### II. Methods of Making Standards

The present standards and methods of use are not limited by the means of generating the standards. In some embodiments, standards are generated by ligating oligonucleotides together to prepare the standards.

Described herein is a method of generating a pool of nucleic acid standards comprising providing multiple copies of at least one sequence of interest comprising nucleic acids; providing a collection of oligonucleotides each comprising a UMI; providing a collection of insertion oligonucleotides of varying lengths; and ligating at least one sequence of interest, at least one oligonucleotide comprising a UMI, and at least one insertion amplicon to produce multiple nucleic acid standards of the pool of nucleic acid standards.

In some embodiments, the at least one sequence of interest and/or insertion oligonucleotide are prepared by amplification.

In some embodiments, the sequence of interest, the oligonucleotides each comprising a UMI, and/or the insertion oligonucleotides comprise a restriction enzyme cleavage site. In some embodiments, the restriction enzyme cleavage site is proximal to the 5′ and/or 3′ end of the sequence of interest, the oligonucleotides each comprising a UMI, and/or the insertion oligonucleotides.

In some embodiments, the method further comprises cleaving the sequence of interest, the oligonucleotides each comprising a UMI, and/or the insertion oligonucleotides with a restriction enzyme before the ligating. In some embodiments, the cleaving with a restriction enzyme produces sticky ends for the ligating. In some embodiments, oligonucleotides comprising a UMI are designed to comprise desired restriction enzyme cleavage sites that are also comprised in the sequence of interest.

Also described herein is a method of generating a pool of nucleic acid standards comprising providing multiple copies of at least one sequence of interest comprising nucleic acids; providing a collection of oligonucleotides each comprising a UMI; and ligating at least one sequence of interest and at least one oligonucleotide comprising a UMI.

In some embodiments, the at least one sequence of interest are prepared by amplification. In some embodiments, the sequence of interest and/or the oligonucleotides each comprising a UMI comprise a restriction enzyme cleavage site. In some embodiments, the restriction enzyme cleavage site is proximal to the 5′ and/or 3′ end of the sequence of interest and/or the oligonucleotides each comprising a UMI.

In some embodiments, the method further comprises cleaving the sequence of interest and/or the oligonucleotides each comprising a UMI with a restriction enzyme before the ligating.

In some embodiments, the cleaving with a restriction enzyme produces sticky ends for the ligating.

In some embodiments, a larger number of UMIs are available compared to the number of LongAmp standards being run. In this way, the number of UMIs is greater than the number of standards being made and duplication of UMIs is minimized.

### III. Methods of Normalizing Amplicon Size Bias

The pool of standards described herein may be used in methods for normalizing amplicon size bias.

Described herein is a method of normalizing amplicon size bias comprising combining a sample comprising a target nucleic acid with a pool of nucleic acid standards of different lengths, wherein each standard comprises a UMI; amplifying the standards and amplicons of a sequence of interest comprised in the target nucleic acid; sequencing the standards and the amplicons of the sequence of interest to generate sequencing data; determining a bias profile based on amplicon size using sequencing data from the standards; and normalizing amplicon size bias using the bias profile.

As used herein, “amplicon size bias” refers to the fact that amplicons of different sizes will amplify differently. In some embodiments, fewer large amplicons are generated as compared with shorter amplicons in a given amplification reaction. In some embodiments, the amplification is PCR amplification. In some embodiments, the amplification is LongAmp PCR.

LongAmp PCR comprises amplification of DNA lengths that cannot typically be amplified using routine PCR methods or reagents. An enzyme optimized for LongAmp PCR may be referred to as a long-range polymerase. Since LongAmp PCR results are improved if a full amplicon is produced, since generation of an incomplete amplicon in a cycle leads to further generation of incomplete amplicons in later PCR cycles. In some embodiments, a long-range polymerase has a high processivity (i.e., incorporates a relatively high number of nucleotides during a single binding event by the DNA polymerase) and/or fast extension rate.

Long-range polymerases with high processivity and fast extension rates help ensure efficient DNA synthesis of long templates and cut down on cycling time. A wide variety of protocols and long-range polymerases are known for use in LongAmp PCR, such as LongAmp Taq DNA polymerase and Phusion DNA polymerase (New England Biolabs). In some embodiments, the long-range polymerase is PrimeSTAR GXL DNA polymerase (Takara).

In some embodiments, amplicon size bias in LongAmp PCR can be normalized with methods using nucleic acid standards described herein. In some embodiments, standards are used to generate a bias profile, wherein this bias profile can be used to normalize data on amplicons generated from a sequence of interest. In some embodiments, the effect of amplicon size on amplification of amplicons from a sequence of interest can be normalized using data generated with the standards described herein.

In some embodiments, amplifying amplicons of the sequence of interest comprises amplifying amplicons from the target nucleic acid with a pair of PCR primers that bind to primer binding sequences at the ends of the sequence of interest. In some embodiments, the standards comprise the same primer binding sequences as those at the ends of the sequence of interest.

In some embodiments, the method further comprises generating a library of fragments after the amplifying and before the sequencing.

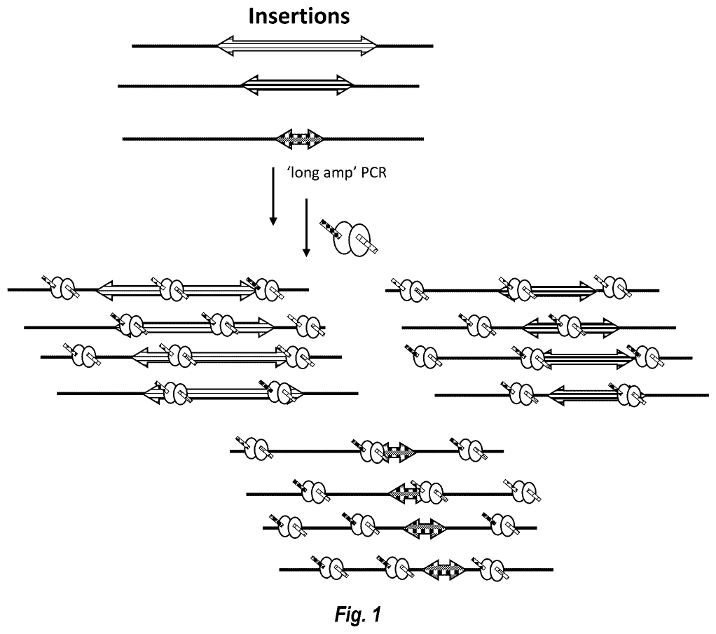

In some embodiments, the generating a library of fragments is by tagmentation. Such a method is shown in FIG. 1, wherein fragments are generated by a Nextera fragmentation protocol. Such a method generates fragments comprising, for example, different insertion mutations (labeled with arrows in FIG. 1). In this ‘long amp’ PCR and fragmentation steps, a pool of standards as described herein could be added for normalizing amplicon size bias during the PCR. In this way, the pool of standards is subjected to the same amplification and fragmentation conditions as the sequence of interest.

In some embodiments, the sequencing data from the standards used to determine the bias profile is the unique molecule count of UMIs comprised in the standards. In other words, one skilled in the art could use standard analysis of sequencing data to determine the number of duplicated UMI from different standards. Since these UMIs originated from standards of different lengths, the count of different UMIs can provide a measure of the efficiency of amplification of different-sized amplicons to generate the bias profile. In this way, the number of amplicons generated for different sequences from the sequence of interest (including amplicons generated from the wild-type sequence of interest and also the sequence of interest comprising indels) can be compared to the bias profile. In other words, the comparison of data generated from the sequence of interest in comparison to the standards can be used to normalize the sequencing data for amplicon size bias. For example, if insertion standards of a similar size as large insertion mutation of the sequence of interest amplified at a 3-times lower rate than standards of a similar size as the wild-type sequence of interest, the user could normalize the number of copies of these large insertion mutations in comparison to the wild-type sequence. Similarly, one skilled in the art could normalize for a larger number of large deletion mutations (i.e., where a large amount of sequence is lost) in comparison to the wild-type sequence using deletion standards.

A. Long Amplification PCR and Sequencing

Long amplification PCR (LongAmp) refers to a PCR reaction that is optimized for long amplicons. Such a LongAmp reaction is shown in FIG. 1 (long amp′ PCR). Such methods of optimized LongAmp PCR are well-known in the art.

In some embodiments, long amplicons may be greater than 5,000 kilobases, greater than 10,000 kilobases, or greater than 20,000 kilobases.

In some embodiments, long amplicons are generated from a sequence of interest that may comprise a large insertion mutation. For example, a long amplicon may be approximately 10,000 kilobases, while the wild-type amplicon from this sequence of interest is approximately 1,000 kilobases.

In some embodiments, LongAmp is used to optimize identification of long insertion mutations in a sequence of interest.

After LongAmp PCR, library preparation may be done before sequencing of the library fragments. For example, tagmentation may be used (such as with Nextera systems from Illumina) for library preparation for sequencing.

In some embodiments, the standards are used to run control assays. In some embodiments, these control assays are separate from LongAmp PCR reactions. In some embodiments, the standards are spiked in a known amount into each LongAmp PCR reaction. By “spiked in,” it is meant that the standards are amplified in the same reaction solution as the LongAmp PCR reaction.

### IV. Methods of Determining DNA Damage in Libraries

Described herein is a quantitative PCR (qPCR) method to quality control (QC) libraries. Such methods can allow a user to determine the amount of DNA damage present in the library before performing further analysis of the library, such as sequencing. In some embodiments, the QC assay differentiates libraries with different levels of damage.

In some embodiments, these libraries can be used for sequencing. In some embodiments, the libraries are intended for long-read sequencing. In some embodiments, libraries are prepared using tagmentation and/or bead-linked transposomes. The present methods of determining DNA damage in libraries can be used with libraries generated by any method.

As used herein, a “library molecule” refers to a single molecule comprised within the library. In some embodiments, each library molecule may comprise a different insert from a target nucleic acid. Library molecules may be generated with standard tagmentation or ligation protocols that are well-known in the art.

Many sequencing applications require the presence of one or more adapter in a library molecule. Often, these adapter sequences are at both ends of inserts. In some embodiments, sequences comprised in adapters are used in sequencing applications, such as to allow for binding of a library molecule to a flowcell or for binding of a sequencing primer to a library molecule. In some embodiments, adapter sequences are required at both ends of inserts for sequencing applications, such as for binding to two different sequencing primer sequences. In such scenarios, library molecules that lack one adapter sequence (such as nicked libraries or amplicons thereof) cannot be successfully sequenced.

In some embodiments, a library comprises long-read hairpin adapter-comprising library molecules. The insert size in long-read library molecules may be 5 kb or greater, 10 kb or greater, 15 kb or greater, 20 kb or greater, 25 kb or greater, or 30 kb or greater. In some embodiments, hairpin adapters can be added to long regions of DNA comprised in inserts within library molecules. In some embodiments, hairpin adapters may be added to inserts using ligation or tagmentation protocols. For example, NEB's NEBNext Multiplex Oligos for Illumina® uses adapter ligation with unique hairpin loop structures that minimize adapter-dimer formation.

In some embodiments, hairpin adapter can be added to inserts during a tagmentation reaction. “Tagmentation,” as used herein, refers to the use of transposase to fragment and tag nucleic acids. Tagmentation includes the modification of DNA by a transposome complex comprising transposase enzyme complexed with one or more tags (such as adaptor sequences) comprising transposon end sequences (referred to herein as transposons). Tagmentation thus can result in the simultaneous fragmentation of the DNA and ligation of the adaptors to the 5′ ends of both strands of duplex fragments. Tagmentation, however, is only one method of generating a library and other methods (such as ligation) can also be used to generate libraries for use with the present QC assay.

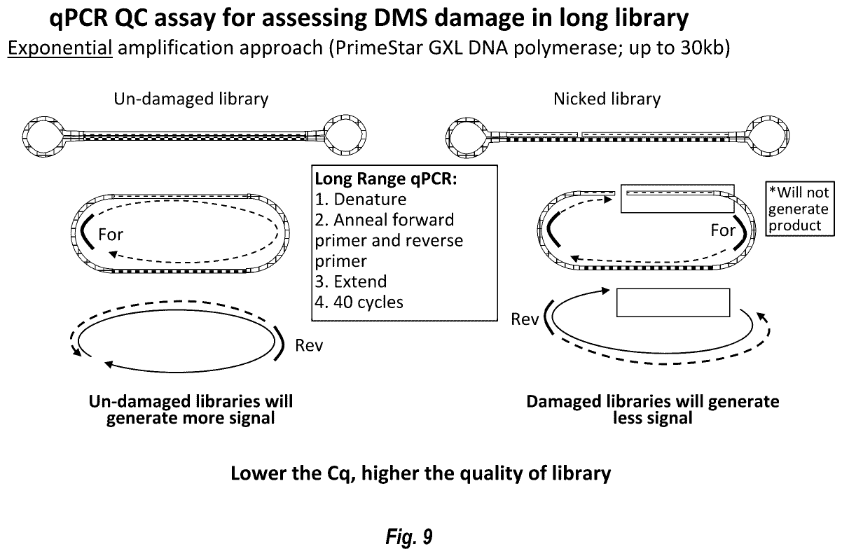

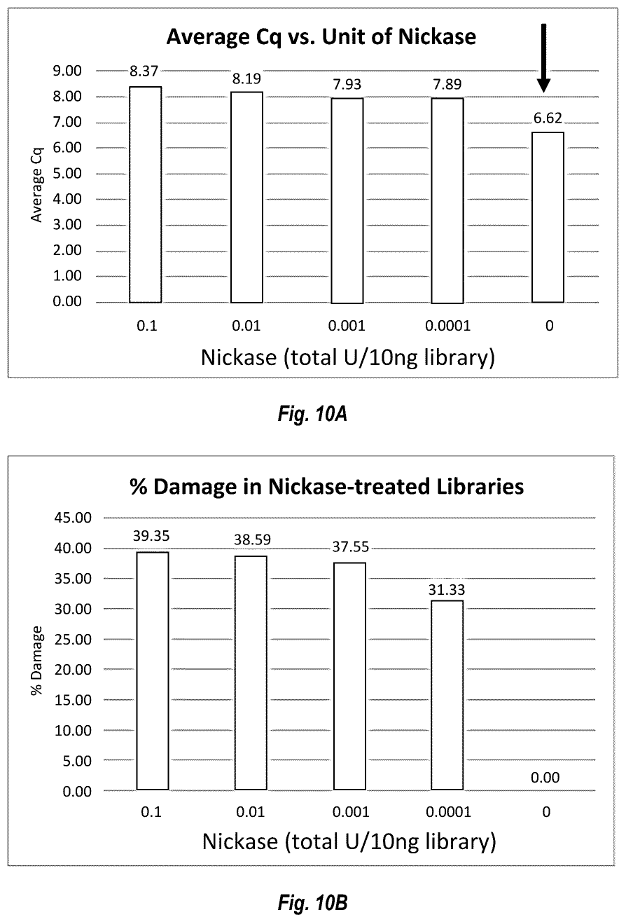

In some embodiments, a method of determining the presence of DNA damage in a library comprising one or more library molecule, wherein each library molecule comprises a double-stranded DNA insert with a hairpin adapter at each end of the insert, comprises denaturing the first stand and second strand of the double-stranded DNA inserts comprised in library molecules; annealing a forward primer and a reverse primer to library molecules; amplifying to produce library amplicons; and assessing the presence of DNA damage based on the number of library amplicons produced. An exemplary method is shown in FIG. 9, which shows that a library molecule with a nick will not generate a full-length amplicon.

The methods described herein may use a long-range polymerase to amplify library molecules for QC. In some embodiments, the QC assay differentiates libraries with different levels of damage, resulting in Cq values that correlate to percentage damage in the library preparation. The presently described method can be applied to any library comprising one or more hairpin adapter, with particular use for long-insert library preparations for long-read sequencing. In some embodiments, use of the present QC assay avoids use of damaged libraries, resulting in a savings of time, money, and consumables.

A. DNA Damage in Libraries

All methods of library preparation can introduce damage to nucleic acids during the preparation process. For example, any pipetting step can lead to shearing of a nucleic acid. While users may take steps to reduce potential damage, this damage cannot be fully avoided or predicted.

Inserts within library molecules may comprise double-stranded nucleic acids obtained as fragments from one or more larger nucleic acid. Fragmentation can be carried out using any of a variety of techniques known in the art including, for example, nebulization, sonication, chemical cleavage, enzymatic cleavage, or physical shearing. However, any of these fragmentation methods has the potential to introduce DNA damage, such as nicking the DNA.

Accordingly, it is important to be able to assess DNA damage in libraries. For example, a user would not want to perform further sequencing on a library with extensive DNA damage, as the sequencing quality would be poor. Similarly, a user might have difficulty determining the proper amount of library product to sequence if much of the library is damaged. For many sequencing platforms, library molecules need adapter sequences at both ends of fragments for uses such as binding to a flowcell or binding to a sequencing primer. In the absence of the proper adapters, such as when a library molecule has DNA damage, a library molecule (and its amplicons) will not generate analyzable sequencing data.

Assessment of DNA damage can allow users to avoid further use of damaged libraries. In this way, users can save time and reagent costs for applications like sequencing if low library quality precludes generation of high-quality data. In some embodiments, libraries with low quality are excluded from sequencing.

In some embodiments, the DNA damage is one or more nick. In some embodiments, one or more nick can be converted into a double-stranded break before a QC assay is performed.

1. Nicks

In some embodiments, the DNA damage comprises one or more nicks in a library molecule. As used herein, the one or more nicks can be a single nick or multiple separate nicks.

In some embodiments, the one or more nicks are within the insert comprised in a library molecule. Since the insert can be a double-stranded insert, a nick refers to a break in one strand of the insert, where a break is not present in the other strand at that position. As used herein, a nick thus can refer to a discontinuity in a double-stranded DNA insert where there is no phosphodiester bond between adjacent nucleotides of one strand. In some embodiments, one or more nick was generated by DNA damage during library preparation. For example, shearing during pipetting may lead to a nick in a library molecule.

In some embodiments, a Cq value generated in a QC assay is greater when a greater percentage of library molecules in the library comprise one or more nicks, as discussed below.

In some embodiments, the DNA damage comprises two or more nicks in a library molecule, wherein the nicks are in the same strand of the double-stranded DNA insert.

In some embodiments, the DNA damage comprises two or more nicks in a library molecule, wherein the nicks are in both strands of the double-stranded DNA insert. When two or more nicks are in different strands, these nicks may be at different positions, to differentiate from double-stranded DNA breaks that are described below.

When a nick is encountered during amplification, the DNA polymerase may be unable to extend the amplicon past the nick. Thus, one or more nick can lead to generation of incomplete amplicons, which do not have the full sequence of the library molecule. In some embodiments, the forward primer and/or the reverse primer cannot generate an amplicon corresponding to the full sequence of the library molecule if the library molecule comprises one or more nicks. Such amplicons without the full sequence of the library molecule may be unsequencable (due to a lack of an adapter sequence that should be at one or both ends of the insert).

In some embodiments, an amplicon generated from a library molecule comprising a nick lacks a sequence for binding to the forward and/or reverse primer.

In some embodiments, library molecules comprising a nick generate fewer amplicons during the amplifying as compared to library molecules not comprising a nick. As discussed below, the present QC methods can estimate the Cq value of library molecules comprising nicks and thus indicate to a user that a library is of relatively low quality (with a high Cq value) or relatively high quality (with a low Cq value). In this way, a Cq value can be used to estimate the quality of a given library for assessing whether to further evaluate the library, such as by sequencing, and to avoid the time and expense associated with sequencing a library that will yield poor data.

2. Double-Stranded DNA Breaks Generated from Nicks

In some embodiments, a method further comprises generating a double-stranded break from a nick. In some embodiments, a double-stranded break is generated from a nick before annealing the forward primer and the reverse primer in a QC method.

In some embodiments, an enzyme is used to prepare a double-stranded break from a nick. In other words, the generating a double-stranded break may be performed using an enzymatic reaction. In some embodiments, the enzymatic reaction is performed by an endonuclease. In some embodiments, the endonuclease is a T7 endonuclease.

In some embodiments, a library molecule comprising a double-stranded break does not generate amplicons corresponding to the full sequence of the library molecule during the amplifying. In some embodiments, the double-stranded break cleaves the library molecule within the insert, and full-length amplicons of the library molecule cannot be generated after the cleavage.

In some embodiments, an amplicon generated from a library molecule comprising a double-stranded break lacks a sequence for binding to the forward and/or reverse primer. In some embodiments, the double-stranded break cleaves the library molecule within the insert, and the primer binding sequences that are comprised in two different hairpin adapters (associated with the two ends of the library insert) are separated. In some embodiments, after cleavage, neither the forward primer nor the reverse primer can generate a full-length amplicon after binding to a library molecule.

B. Hairpin Adapters

As used herein, a “hairpin” refers to a nucleic acid comprising a pair of nucleic acid sequences that are at least partially complementary to each other. These two nucleic acid sequences that are at least partially complementary can bind to each other and mediate folding of a nucleic acid. In some embodiments, the two nucleic acid sequences that are at least partially complementary generate a nucleic acid with a hairpin secondary structure.

A “hairpin adaptor,” as used herein, refers to an adaptor that comprises at least one pair of nucleic acid sequences that are at least partially complementary to each other. In some embodiments, a hairpin adaptor has a folded secondary structure.

In some embodiments, a hairpin adapter comprises one or more adapter sequence. In some embodiments, the adaptor sequence comprises a primer sequence, an index tag sequence, a capture sequence, a barcode sequence, a cleavage sequence, or a sequencing-related sequence, or a combination thereof. As used herein, a sequencing-related sequence may be any sequence related to a later sequencing step. A sequencing-related sequence may work to simplify downstream sequencing steps. For example, a sequencing-related sequence may be a sequence that would otherwise be incorporated via a step of ligating an adaptor to nucleic acid fragments. In some embodiments, the adaptor sequence comprises a P5 or P7 sequence (or their complement) to facilitate binding to a flow cell in certain sequencing methods.

In some embodiments, a hairpin adaptor comprises an amplification primer sequence (i.e., a sequence that binds to an amplification primer). In some embodiments, a hairpin adaptor comprises an amplification primer sequence and all or part a sequence at least partially complementary to the adaptor sequence. In some embodiments, the amplification primer sequence comprised in the hairpin is a universal primer sequence. A universal sequence is a region of nucleotide sequence that is common to, i.e., shared by, two or more nucleic acid molecules.

In some embodiments, either the forward primer or the reverse primer binds to one or more sequences comprised in one or both hairpin adapter. In some embodiments, both the forward primer and the reverse primer bind to one or more sequences comprised in one or both hairpin adapter. In some embodiments, the forward primer binds to a sequence comprised in the hairpin adapter attached to a first end of the double-stranded DNA insert, and the reverse primer binds to a sequence comprised in the hairpin adapter attached to a second end of the double-stranded DNA insert.

In some embodiments, library molecules comprise an insert comprising double-stranded nucleic acid and a hairpin adaptor at both ends of the insert. In some embodiments, the insert comprises a fragment from a target nucleic acid. Methods of incorporating hairpin adapters are well-known in the art, such as by ligation or tagmentation.

For example, NEBNext® Multiplex Oligos for Illumina® (New England BioLabs) provides hairpin adapters and primers to increase yield of library products. In some embodiments, hairpin adapters include hairpin loop structures that minimize adapter-dimer formation. In some embodiments, hairpin adapters are ligated to end-repaired, dA-tailed DNA. In some embodiments, a hairpin adapter comprises a loop containing a uracil, which is removed by treatment with a USER reagent. In some embodiments, the USER Enzyme is a mix of uracil DNA glycosylase (UDG) and a DNA glycosylase-lyase (such as Endonuclease VIII). In some embodiments, USER treatment can open up the loop of a hairpin adapter and make it available as a substrate for amplification to incorporate index primers and subsequent sequencing.

In some embodiments, a hairpin adapter is incorporated using locus-specific primers and USER reagents to generate overhangs for ligating hairpin adapters. An exemplary method would be SMRTbell library preparation (Pacific Biosciences, see SMRTbell Library Preparation & SMRT Sequencing Workflow Updates, 2017).

In some embodiments, hairpin adapters are comprised in library molecules with relatively large inserts, wherein the library molecules are designed for long-read sequencing.

In some embodiments, each hairpin adapter comprises an amplification primer binding site. In some embodiments, the hairpin adapter at a first end of an insert comprises a different amplification primer binding site than the hairpin adapter at a second end of an insert. In some embodiments, the hairpin adapter at a first end of an insert comprises a first amplification primer binding site and the hairpin adapter at a second end of an insert comprises a second amplification primer binding site. In some embodiments, the first amplification primer binding site and the second amplification primer binding site mediate amplification in opposite directions.

In some embodiments, such as that shown in FIG. 9, a hairpin adapter at a first end of an insert may comprise a forward amplification primer binding site and a hairpin adapter at a second end of an insert may comprise a reverse amplification primer binding site.

C. Amplification

In some embodiments, the method further comprises amplifying library molecules using an amplification primer that binds to an amplification primer sequence. In some embodiments, one or both hairpin adapters comprised in library molecules comprises an amplification primer.

In some embodiments, the amplifying is optimized for amplifying library molecules that are 5 kb or greater, 10 kb or greater, 15 kb or greater, 20 kb or greater, 25 kb or greater, or 30 kb or greater.

In some embodiments, the amplifying is performed with a polymerase optimized for amplification of long amplicons. In some embodiments, the polymerase is optimized for amplification of amplicons of 20 kb or more or 30 kb or more.

A number of exemplary polymerases optimized for amplification of long amplicons are known in the art. One exemplary polymerase would be PrimeSTAR GXL DNA polymerase (Takara).

In some embodiments, the polymerase has a higher processivity and/or extension rate as compared to a wildtype Taq polymerase. In some embodiments, the polymerase comprises one or more mutation or fusion that increase processivity or extension rate.

As used herein, “processivity” of a polymerase refers to the number of nucleotides that a polymerase can incorporate into DNA during a single template-binding event, before dissociating from a DNA template. Accordingly, a polymerase with relatively high processivity can incorporate a large number of nucleotides during a single template-binding event. Higher processivity can increase the likelihood that a full amplicon is generated during a PCR cycle.

As used herein, “extension rate” of a polymerase is the number of nucleotides that it can incorporate into DNA over a period of time. In some embodiments, a polymerase with a relatively high extension rate can generate a full amplicon of a library molecule during a PCR cycle. In some embodiments, a polymerase has an extension rate of 2 kb/min or greater, 3 kb/minute or greater, or 4 kb/minute or greater.

In some embodiments, the polymerase has an extension rate of 3 kb/minute or greater.

In some embodiments, the amplifying is exponential.

In some embodiments, 30 or more or 40 or more cycles of amplifying are performed.

In some embodiments, amplification primers may comprise index sequences. These index sequences may be used to identify the sample and location in the array. In some embodiments, an index sequence comprises a unique molecular identifier (UMI). UMIs are described in Patent Application Nos. WO 2016/176091, WO 2018/197950, WO 2018/197945, WO 2018/200380, and WO 2018/204423, each of which is incorporated herein by reference in its entirety.

In some embodiments, samples are amplified on a solid support.

For example, in some embodiments, samples are amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to herein as “clustered arrays”. The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, in some embodiments via a covalent attachment. Cluster amplification methodologies are examples of methods wherein an immobilized nucleic acid template is used to produce immobilized amplicons. Other suitable methodologies can also be used to produce immobilized amplicons from immobilized DNA fragments produced according to the methods provided herein. For example, one or more clusters or colonies can be formed via solid-phase PCR whether one or both primers of each pair of amplification primers are immobilized.

In other embodiments, samples are amplified in solution. For example, in some embodiments, samples are cleaved or otherwise liberated from a solid support and amplification primers are then hybridized in solution to the liberated molecules. In other embodiments, amplification primers are hybridized to desired samples for one or more initial amplification steps, followed by subsequent amplification steps in solution. In some embodiments, an immobilized nucleic acid template can be used to produce solution-phase amplicons.

It will be appreciated that any of the amplification methodologies described herein or generally known in the art can be utilized with universal or target-specific primers to amplify desired samples. Suitable methods for amplification include, but are not limited to, the polymerase chain reaction (PCR), strand displacement amplification (SDA), transcription mediated amplification (TMA) and nucleic acid sequence-based amplification (NASBA), as described in U.S. Pat. No. 8,003,354, which is incorporated herein by reference in its entirety. The above amplification methods can be employed to amplify one or more nucleic acids of interest. For example, PCR, including multiplex PCR, SDA, TMA, NASBA and the like can be utilized to amplify immobilized DNA fragments. In some embodiments, primers directed specifically to the nucleic acid of interest are included in the amplification reaction.

Other suitable methods for amplification of nucleic acids can include oligonucleotide extension and ligation, rolling circle amplification (RCA) (Lizardi et al., Nat. Genet. 19:225-232 (1998), which is incorporated herein by reference) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835, all of which are incorporated by reference) technologies. It will be appreciated that these amplification methodologies can be designed to amplify immobilized DNA fragments. For example, in some embodiments, the amplification method can include ligation probe amplification or oligonucleotide ligation assay (OLA) reactions that contain primers directed specifically to the nucleic acid of interest. In some embodiments, the amplification method can include a primer extension-ligation reaction that contains primers directed specifically to the nucleic acid of interest. As a non-limiting example of primer extension and ligation primers that can be specifically designed to amplify a nucleic acid of interest, the amplification can include primers used for the GoldenGate assay (Illumina, Inc., San Diego, CA) as exemplified by U.S. Pat. Nos. 7,582,420 and 7,611,869, each of which is incorporated herein by reference in its entirety.

Exemplary isothermal amplification methods that can be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587, each of which is incorporated herein by reference in its entirety. Other non-PCR-based methods that can be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyperbranched strand displacement amplification which is described in, for example Lage et al., Genome Research 13:294-307 (2003), each of which is incorporated herein by reference in its entirety. Isothermal amplification methods can be used with the strand-displacing Phi 29 polymerase or Bst DNA polymerase large fragment, 5′->3′ exo-for random primer amplification of genomic DNA. The use of these polymerases takes advantage of their high processivity and strand displacing activity. High processivity allows the polymerases to produce fragments that are 10-20 kb in length. As set forth above, smaller fragments can be produced under isothermal conditions using polymerases having low processivity and strand-displacing activity such as Klenow polymerase. Additional description of amplification reactions, conditions and components are set forth in detail in the disclosure of U.S. Pat. No. 7,670,810, which is incorporated herein by reference in its entirety.

D. Sequencing

In some embodiments, the method further comprises sequencing of library products and amplified library products (i.e., amplicons). In some embodiments, the analysis of libraries after the QC assay is sequencing.

In some embodiments, a method comprises determining conditions for analysis of the library based on the Cq value. In some embodiments, the QC assay is used to determine conditions for sequencing a library. In some embodiments, the QC assay is used to determine that a given library should not be sequenced. For example, the QC assay may estimate that there are not enough library molecules in a given library, such that sequencing data generated from the library would be of low quality.

In some embodiments, the method allows sequencing of the full sequence of the insert.

One exemplary sequencing methodology is sequencing-by-synthesis (SBS). In SBS, extension of a nucleic acid primer along a nucleic acid template is monitored to determine the sequence of nucleotides in the template. The underlying chemical process can be polymerization (e.g. as catalyzed by a polymerase enzyme). In a particular polymerase-based SBS embodiment, fluorescently labeled nucleotides are added to a primer (thereby extending the primer) in a template dependent fashion such that detection of the order and type of nucleotides added to the primer can be used to determine the sequence of the template.

Flow cells provide a convenient solid support for sequencing. For example, to initiate a first SBS cycle, one or more labeled nucleotides, DNA polymerase, etc., can be flowed into/through a flow cell that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be adapted for application of pyrosequencing to amplicons produced according to the present disclosure are described, for example, in WIPO Pat. App. Pub. No. WO 2012058096, US 2005/0191698 A1, U.S. Pat. Nos. 7,595,883, and 7,244,559, each of which is incorporated herein by reference.

Some embodiments can utilize methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying nucleic acids using kinetic exclusion can be readily applied to substrates used for detecting protons. More specifically, methods set forth herein can be used to produce clonal populations of amplicons that are used to detect protons.

Another useful sequencing technique is nanopore sequencing (see, for example, Deamer et al. Trends Biotechnol. 18, 147-151 (2000); Deamer et al. Acc. Chem. Res. 35:817-825 (2002); Li et al. Nat. Mater. 2:611-615 (2003), the disclosures of which are incorporated herein by reference). In some nanopore embodiments, the nucleic acid or individual nucleotides removed from a nucleic acid pass through a nanopore. As the nucleic acid or nucleotide passes through the nanopore, each nucleotide type can be identified by measuring fluctuations in the electrical conductance of the pore. (U.S. Pat. No. 7,001,792; Soni et al. Clin. Chem. 53, 1996-2001 (2007); Healy, Nanomed. 2, 459-481 (2007); Cockroft et al. J. Am. Chem. Soc. 130, 818-820 (2008), the disclosures of which are incorporated herein by reference).

Exemplary methods for array-based expression and genotyping analysis that can be applied to detection according to the present disclosure are described in U.S. Pat. Nos. 7,582,420; 6,890,741; 6,913,884 or 6,355,431 or US Pat. Pub. Nos. 2005/0053980 A1; 2009/0186349 A1 or US 2005/0181440 A1, each of which is incorporated herein by reference.

An advantage of the methods set forth herein is that they provide for rapid and efficient detection of a plurality of nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines, and the like. A flow cell can be configured and/or used in an integrated system for detection of nucleic acids. Exemplary flow cells are described, for example, in US 2010/0111768 A1 and US Pub. No. 2012/0270305 A1, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in US Pub. No. 2012/0270305, which is incorporated herein by reference.

E. Cq values

In some embodiments, the number of library amplicons produced is estimated by quantitative PCR (qPCR). In some embodiments, the number of library amplicons produced is estimated by measuring a cycle of quantification (Cq, also known as quantification cycle) value.

As used herein, the Cq value is the PCR cycle number at which a sample's reaction curve intersects the threshold line. Thus, the Cq value indicates how many cycles of PCR were needed to detect a signal above noise for a given sample.

This may be determined with fluorescent dyes and probes, and the method measures the number of amplification cycles needed to detect the fluorescence. Using this method a Cq value is the cycle number at which the fluorescence of a PCR product can be detected above background signal. Accordingly, a higher Cq value indicates that less nucleic acid is present in the sample.

As described in Bustin et al., Clinical Chemistry 55(4):611-622 (2009), the terms threshold cycle (Ct), crossing point (Cp), and take-off point (TOP) all refer to the same measurement as a Cq value, and the differences in nomenclature are simply based on different instrumentation. All of these terms (Ct, Cp, and TOP) refer to method of determining the PCR cycle number at which a sample's reaction curve intersects the threshold line, and accordingly all these values are synonyms for a Cq value.

In some embodiments, a higher number of library amplicons results in a lower Cq value. In some embodiments, a library with a lower Cq value has less DNA damage. In some embodiments, a library with less DNA damage will produce better sequencing results.

In some embodiments, those library products comprising a nick will not generate an amplicon corresponding to the full sequence of the library molecule. In some embodiments, extension during an amplification cycle (i.e., generation of an amplicon) stops at the site of a nick in the library molecule.

For example, FIG. 9 shows how a library molecule with a nick (i.e., a damaged library) will generate less signal since amplification does not produce a full sequence of the library molecule with both the forward and reverse amplification primer binding sites.

In some embodiments, Cq values correlate to the percentage of damage in the library. In some embodiments, the damage was introduced during library preparation.

In some embodiments, high Cq values correlate with more DNA damage of library molecules. In some embodiments, libraries with high Cq values show lower sequencing performance. In some embodiments, the lower sequencing performance is measured by total output (Gb) or percentage P1.

In some embodiments, Cq values that are atypically low (e.g. lower than 2.58) may also have lower sequencing performance.

In some embodiments, a desired Cq range may be determined that generates sequencing runs with adequate data quality depending on the next use for the library. In some embodiments, a desired Cq range may be from 2.58-5. The Cq range may vary based on the specific type of libraries being used. Accordingly, a user might run initial studies to determine a desired Cq range that results in sequencing data of sufficient quality, and then choose to only sequence libraries having Cq values within this range. Such analysis to determine a desired Cq range is easily performed by one skilled in the art, and such determination would not be considered an undue burden.

F. Long-Read Sequencing

Standard short-read sequencing provides accurate base level sequence to provide short range information, but short-read sequencing may not provide long range genomic information. Further, because haplotype information is not retained for the sequenced genome or the reference with short read data, the reconstruction of long-range haplotypes is challenging with standard methods. As such, standard sequencing and analysis approaches generally can call single nucleotide variants (SNVs), but these methods may not identify the full spectrum of structural variation seen in an individual genome. “Structural variations” of a genome, as used herein, refers to events larger than a SNV, including events of 50 base pairs or more. Representative structural variants include copy-number variations, inversions, deletions, and duplications.

“Linked long read sequencing” or “linked-read sequencing” refers to sequencing methods that provide long range information on genomic sequences.

In some embodiments, linked-read sequencing uses molecular barcodes to tag reads that come from the same long DNA fragment. When unique barcodes are added to every read generated from an individual DNA molecule, the reads can that DNA molecule can be linked together. In other words, reads that share a barcode can be grouped as deriving from a single long input molecule allowing long range information to be assembled from short reads.

In some embodiments, linked-read sequencing can be used for haplotype reconstruction. In some embodiments, linked-read sequencing improves calling of structural variants. In some embodiments, linked-read sequencing improves access to region of the genome with limited accessibility. In some embodiments, linked-read sequencing is used for de novo diploid assembly. In some embodiments, linked-read sequencing improves sequencing of highly polymorphic sequences (such as human leukocyte antigen genes) that require de novo assembly.

In some embodiments, the sequencing is long-read sequencing of library molecules that are 5 kb or greater, 10 kb or greater, 15 kb or greater, 20 kb or greater, 25 kb or greater, or 30 kb or greater.

G. Methods Comprising Preparation of Double-Stranded DNA Breaks

In some embodiments, nicks are converted into double-stranded DNA breaks. An advantage of generating double-stranded DNA breaks from nicks is that no amplicons corresponding to a full library molecule can be generated after a double-stranded break is generated in a library product. In this way, library molecules that comprised nicks will not generate any amplicons corresponding to the full sequence of the library product. In contrast, a nicked library molecule comprising a nick in a single strand of the double-stranded insert generates fewer amplicons, but can generate some amplicons corresponding to the full sequence of the library product (as shown in FIG. 9). This is because either the reverse or forward primer could produce an amplicon corresponding to the full sequence of the library molecule.

An advantage of generating a double-stranded break from a nick is that a library molecule with a double-stranded break cannot generate any full-length amplicons with both the binding site with the forward and reverse primer.

In some embodiments, nicks are converted into double-stranded breaks using an endonuclease. In some embodiments, the endonuclease is a mutant T7 endonuclease. In some embodiments, the mutant endonuclease is a maltose binding protein (MBP)-T7 Endo I. In some embodiments, a T7 endonuclease produces counter nicks, in order to generate a double-stranded break in the DNA where a nick had previously been located in a single strand. Such generation of a double-stranded break from a nick may be termed cleaving across a nick.

H. Methods with SMRTbell Templates

In some embodiments, library molecules comprise two hairpin adapters that are ligated to ends of a double-stranded DNA fragment. In some embodiments, such adapters form a closed loop.

While the present invention is not limited to this preparation method, in some embodiments, the library molecules are SMRTbell templates. SMRTbell templates are well-known in the field for use with single-molecule real-time (SMRT) sequencing. In some embodiments, SMRT sequencing uses methodologies from Pacific Biosciences (PacBio) (See, for example, Rhoads and Au, Genomics Proteomics Bioinformatics 13:278-289 (2015)). As used herein, SMRT sequencing and PacBio sequencing may be used interchangeably.

SMRT sequencing technology utilizes circular consensus sequencing (CCS) to generate highly accurate, long high fidelity reads with >99% accuracy and >3 passes. In order to generate the highest output of HiFi reads per sequencing run, high quality SMRTbell templates should be generated that can allow for constant rolling circle amplification (RCA). For example, the PacBio Sequel system can use on-platform RCA to sequence hairpin adapter-ligated library molecules. Therefore, in order to generate CCS reads, the polymerase should sequence in repeated passes to generate long polymerase read-lengths ≥3 times of the length of the insert.

For the polymerase in the SMRT system to sequence efficiently, the input library must be of high quality. During the library preparation process, damage can be introduced to the DNA, either by pipetting, storage or other handling and/or technique errors. If nicked SMRTbell templates are loaded onto the Bio Sequel system for sequencing, the polymerase will fall off at the nick site and terminate RCA, and as a result, the percentage P1 will decrease along with the CCS output from that sequencing run.

An advantage of SMRT sequencing is longer read lengths and faster runs that certain other sequencing methods. For example, PacBio systems are known to be able to generate read lengths of over 60 kilobases. These longer read lengths can allow for the precise location and sequence of repetitive regions within a single read, which might not be available with other sequencing platforms.

In summary, SMRT sequencing is known to have lower throughput, higher error rate, and higher cost per base than some other methods, and users would want to minimize these disadvantages. In some embodiments, the present methods of quality control for libraries allows a user to select libraries for sequencing that have a high likelihood to generate sequencing runs of sufficient quality with methods such as SMRT sequencing. In this way, a user can avoid the expense and time spent in sequencing runs that had DNA damage that limited the ability to generate quality sequencing data.

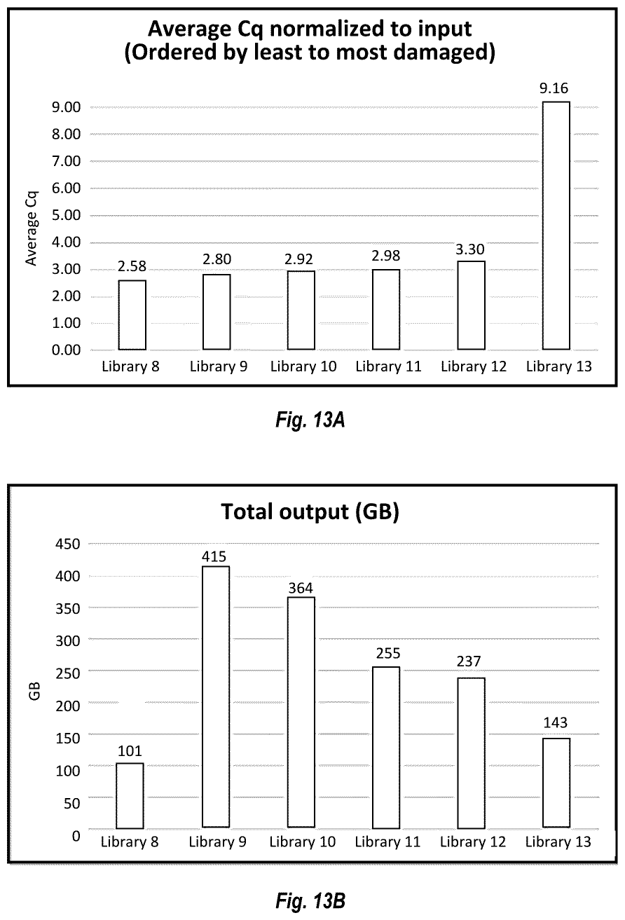

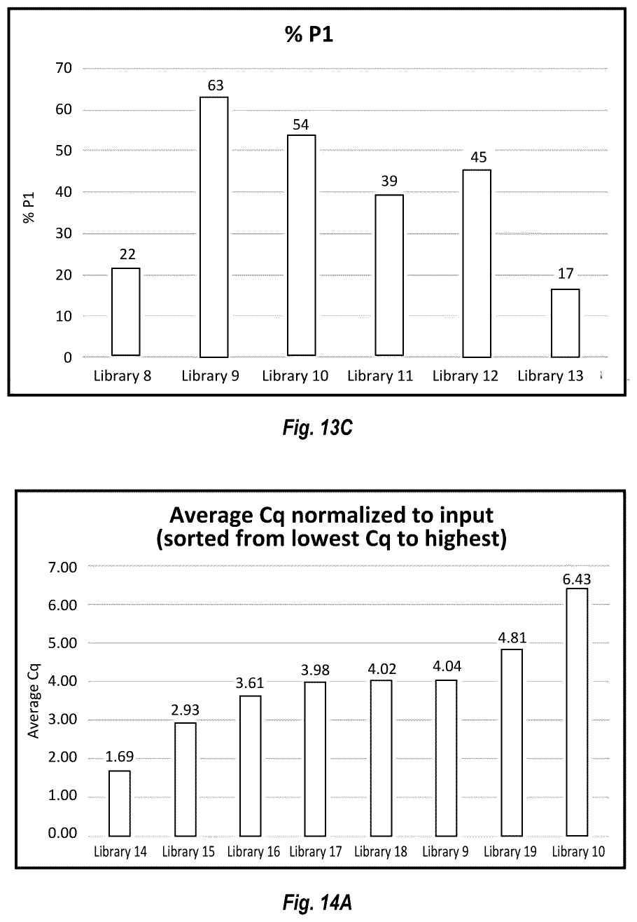

In some embodiments, QC methods described herein maximize the percentage P1 and total output from a SMRT sequencing run. In some embodiments, the qPCR QC method described herein allows customers to avoid loading damaged libraries onto the SMRT sequencing platforms, and therefore to save time, money, reagents, and consumables. FIGS. 13A-15C show some representative data for QC assays with SMRT sequencing.

### V. Method of Determining DNA Damage Using Fluorescence

The amount of DNA damage in a sample comprising DNA can also be measured using fluorescence by methods described herein. In some embodiments, DNA damage can be quantified in a sample DNA using fluorescence before a library is prepared. Such a workflow may be very attractive to allow a user to determine whether there is too much DNA damage in a sample, which would be detrimental to downstream assays like sequencing. For example, a user may quantify DNA damage in a sample and then only prepare a library from the sample if there is a low level (such as 5% or less) of DNA damage. In this way, the user can save time and resources by not preparing a library from a sample with moderate (such as greater than 5%) levels of DNA damage.

In some embodiments, a method of quantifying DNA damage in a sample comprising DNA using fluorescence comprises:
a. combining:
   i. an aliquot of a sample comprising DNA,
   ii. one or more DNA repair enzyme; and
   iii. dNTPs, wherein one or more dNTP is fluorescently labeled;
b. preparing repaired DNA;
c. dephosphorylating the phosphates from dNTPs;
d. binding the repaired DNA to carboxylate or cellulose beads;
e. eluting the bound repaired DNA from the carboxylate or cellulose beads with a resuspension buffer; and
f. measuring fluorescence of the repaired DNA to determine the amount of DNA damage.

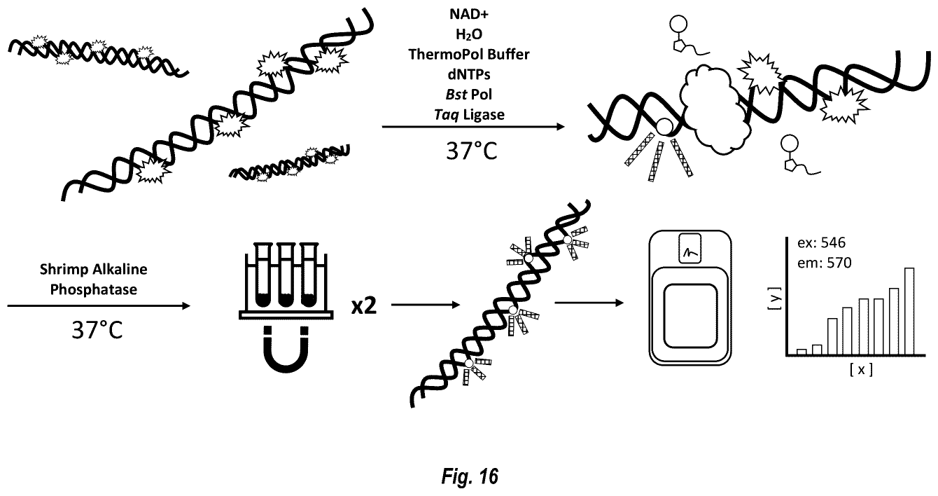

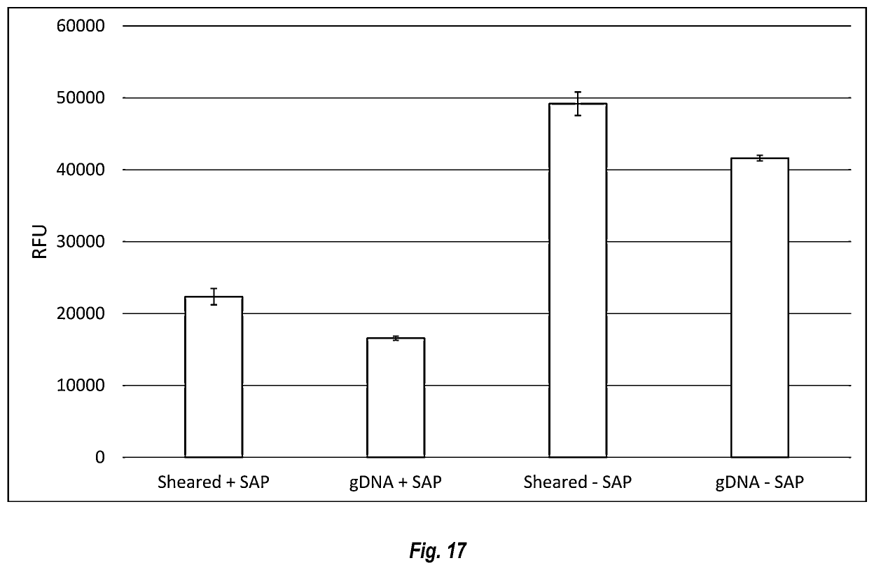

An overview of the method of quantifying DNA damage is shown in FIG. 16, with results of representative experiments using the method shown in FIGS. 17-21.

In some embodiments, a greater fluorescence of the repaired DNA indicates greater DNA damage. In other words, more fluorescently labeled dNTPs will be incorporated if there is a higher level of DNA damage.

In some embodiments, the fluorescence of the repaired DNA is linear over a range difference amounts of DNA damage. In this way, the dynamic range (i.e., the total range of DNA damage that can be accurately measured) of the assay is improved, so the user can evaluate relative differences in damage for various libraries. In some embodiments, a broad linear range may be helpful to accurately determine relatively small amounts of DNA damage if a user is evaluating samples for sensitive downstream assays wherein this amount of DNA damage could negatively impact results.

In some embodiments, the method can assess DNA damage in an aliquot of the sample. In other words, a user may take a small amount of a sample, quantify DNA damage and then potentially perform more assays (such as library preparation or sequencing) based on the results of the quantification of DNA damage.

In some embodiments, the method can assess DNA damage induced by a manipulation of the sample by assessing an aliquot of the same sample before and after the manipulation. In this way, the user can directly measure any DNA damage induced by the manipulation.

In some embodiments, the manipulation is sequencing of a sample. For example, a user may wish to evaluate the impact of different sequencing reagents on a sample comprising DNA to determine if certain reagents induce DNA damage.

In some embodiments, measuring fluorescence of the repaired DNA comprises preparing a standard curve of dilutions of repaired DNA and measuring the fluorescence of the dilutions of repaired DNA. In some embodiments, use of a standard curve can increase the dynamic range of the assay to allow for quantification of small amount of DNA damage. Such a methodology to quantify small amounts of DNA damage may be useful when even a small amount of DNA damage may be detrimental to results of downstream assays (such as sequencing).

In some embodiments, measuring fluorescence of the repaired DNA comprises comparing the fluorescence of the repaired DNA against a separate standard curve of dilutions of only the one or more dNTP that is fluorescently labeled to determine the number of fluorescent dye molecules comprised in the repaired DNA.

In some embodiments, a method further comprises calculating the normalized number of fluorescent dye molecules comprised in the repaired DNA by dividing the number of fluorescent dye molecules determined by the mass of the repaired DNA. Such a measure can estimate what percentage of the DNA is damaged.

In some embodiments, the DNA is genomic DNA, cDNA, or a library comprising fragmented double-stranded DNA. If the DNA is genomic DNA or cDNA, the method may be performed before library preparation.

In some embodiments, the DNA is genomic DNA or cDNA, and the method further comprises preparing a library after determining the amount of DNA damage.

In some embodiments, a library is prepared if the amount of DNA damage is 5% or less, 4% or less, 3% or less, 2% or less, or 1% or less of total nucleotides. In other words, a library may be prepared if the DNA damage is determined to be low. The amount of DNA damage that is acceptable for preparing a library or other downstream assay will depend upon the sensitivity of the downstream assay and the type of DNA damage. For example, short read sequencing may give acceptable sequencing results even with moderate levels of DNA damage (e.g. 5% or less). In contrast, long read sequencing may require lower levels of DNA damage (e.g., 2% or less) for acceptable results and may also be more sensitive to damage induced by nicking.

In some embodiments, if the present assay determines the presence of certain types of damage (such as nicking), this damage may be repaired before further steps such as library preparation or sequencing.

In some embodiments, a library is not prepared if the amount of DNA damage is 5% or greater, 4% or greater, 3% or greater, 2% or greater, or 1% or greater of total nucleotides. In this way, the user avoids wasting time and resources on preparing libraries (and performing further downstream assays like sequencing) if there is a level of DNA damage that would negatively affect results of downstream assays.

In some embodiments, more than one round of binding the repaired DNA to carboxylate or cellulose beads and eluting is performed before measuring the fluorescence. In some embodiments, multiple rounds of bead-based purification improve results of the method. In some embodiments, multiple rounds of bead-based purification reduce non-specific signal. In some embodiments, multiple rounds of bead-based purification two rounds of binding the repaired DNA to carboxylate or cellulose beads and eluting is performed before measuring the fluorescence.

Carboxylate beads (such as SPRI beads) and cellulose beads are commercially available for DNA purification and size selection uses, and such beads may be used in the present method.

In some embodiments, the carboxylate or cellulose beads are magnetic. This property may help with washing of beads after binding of repaired DNA.

In some embodiments, the preparing of repaired DNA is performed at 37° C. In some embodiments, the preparing repaired DNA is performed for 10 minutes or more, 20 minutes or more, 30 minutes or more, 45 minutes or more, or 60 minutes or more.

In some embodiments, dephosphorylating the phosphates from dNTPs reduces nonspecific binding of dNTPs and improves assay results.

In some embodiments, dephosphorylating the phosphates from dNTPs is performed with an enzyme. In some embodiments, the enzyme for dephosphorylating the phosphates from dNTPs is shrimp alkaline phosphatase (SAP) or calf intestinal alkaline phosphatase (CIP).

A variety of different DNA repair enzymes can be used in this method, and as used herein “DNA damage” may refer to multiple different types of DNA modifications (for example nicks and thymine dimers) that may be present in DNA comprised in a single sample.

In some embodiments, the one or more DNA repair enzyme comprises a DNA polymerase. In some embodiments, the DNA polymerase has 5′-3′ polymerase activity but lacks 5′-3′ exonuclease activity. In some embodiments, the DNA polymerase is Bst DNA polymerase, large fragment. In some embodiments, the one or more DNA repair enzyme comprises a ligase. In some embodiments, the ligase is Taq ligase. In some embodiments, the DNA damage comprises a nick in double-stranded DNA.

In some embodiments, the one or more DNA repair enzyme comprises T4 pyrimidine dimer glycosylase (PDG). In some embodiments, the DNA damage comprises a thymine dimer. In some embodiments, the thymine dimer was induced by ultraviolet irradiation.

In some embodiments, the one or more DNA repair enzyme comprises uracil DNA glycosylase (UDG) and an apurinic or apyrimidinic site lyase. In some embodiments, the DNA damage comprises a uracil.

In some embodiments, the one or more DNA repair enzyme comprises formamidopyrimidine DNA glycosylase (FPG) and an apurinic or apyrimidinic site lyase. In some embodiments, the DNA damage comprises an oxidized base.

In some embodiments, more than one DNA repair enzyme is used. In some embodiments, the one or more DNA repair enzyme is a mixture of multiple DNA repair enzymes. Such an approach may be used if a user suspects that the DNA damage may comprise more than one type of damaging modification to the DNA (i.e., thymine dimers and nicks or any other combination of modifications).

In some embodiments, the dNTPs comprise dATP, dGTP, dCTP, and dTTP or dUTP. Any or all the dNTPs may be fluorescently labeled. In some embodiments, all the dNTPs are fluorescently labeled. In some embodiments, dUTP and dCTP are fluorescently labeled.

Any suitable fluorescent label may be comprised in the dNTP. In some embodiments, the fluorescent label is Alexa Fluor 488, Alexa Fluor 546, Alexa Fluor 555, Alexa Fluor 633, fluorescein isothiocyanate (FITC), or tetramethylrhodamine-6)-isothiocyanate (TRITC), although a range of other fluorescent labels across the excitation spectrum may be used. In some embodiments, the fluorescent label has an excitation wavelength that does not damage DNA.

### EXAMPLES

### Example 1. Normalizing Amplicon Size Bias of LongAmp PCR Reaction Using Standards

FIG. 1 presents a representative LongAmp PCR reaction that is then followed by fragmentation, such as with a Nextera product (Illumina). As described herein, a pool of nucleic acid standards of different lengths can be used to normalize for amplicon size bias in this experiment.

Long amplification PCR can be done to generate amplicons from a sequence of interest contained in a target nucleic acid fragment within a sample (as shown in FIG. 1). The sample may be a sample comprised of nucleic acid that has been subjected to gene editing, wherein the user expects that there may be a number of different types of indel mutations.

During this PCR reaction, a pool of nucleic acid standards of different lengths, as described herein, can be included in the reaction. This pool may comprise full-length standards (such as those shown in FIG. 8A), insertion standards (such as those shown in FIG. 8B), and deletion standards (such as those shown in FIG. 8C). In this way, the standards will be amplified under the same conditions as the sequence of interest.

A representative method of making insertion standards is as follows:
Step 1) The oligonucleotide shown in FIG. 2A comprising an N18 UMI is digested using restriction enzymes that cut at restriction site 3 (RS3) and restriction site 4 (RS4);
Step 2) PCR product of FIG. 3 is digested by restriction enzymes that cut at restriction site 1 (RS1) and restriction site 2 (RS2);
Step 3) PCR product of FIG. 4 is digested by RS1 and RS2;
Step 4) Products from steps 2 and 3 are ligated;
Step 5) PCR product of FIG. 4 is digested by RS3 and RS4; and
Step 6) Product from step 5 is ligated with product of step 1.

These steps to prepare insertion standards are expected to generate the products shown in FIG. 2B. The order of the RS digestions is not fixed. Further, if all the restriction enzymes that digest at the RS's are buffer-compatible, all digestion steps may be combined. Alternatively, digestion steps may be performed in separate steps. The ligation steps (steps 4 and 6) may also be combined as a final step in the method of preparing insertion standards.

A representative method of making deletion standards is as follows:
Step 1) The oligonucleotide shown in FIG. 6A (which is identical to the oligonucleotide shown in FIG. 2A and which comprises an N18 UMI) is digested by RS3 and RS4;
Step 2) PCR product of FIG. 5 is digested by RS3 and RS4; and
Step 3) Product of step 2 is ligated with product of step 1.

These steps to prepare deletion standards are expected to generate the products shown in FIG. 6B.

After amplification of the sequence of interest together with the standards, the amplicons (from standards and from the sequence of interest) may then be subjected to a method for preparing a sequencing library. FIG. 1A shows that this may be Nextera fragmentation (i.e. tagmentation), wherein the transposases incorporate adapter sequences at both ends of fragments. The fragments may then be sequenced using sequences that are contained in these adapter sequences (such as sequencing primer binding sites).

The library (comprised of fragments generated from the sequence of interest and the standards) can then be sequenced. Using the UMIs contained in the individual standards, a bias profile can be generated. This bias profile would account for the fact that larger standards have fewer unique replicates, because replicates of a given standard can be identified using the standard's UMI. These data can be used to normalize amplicon size bias. In this way, the user can approximate how many original copies of the sequence of interest had a given indel mutation. In other words, the method can control for that fact that large insertion mutations of the sequence of interest (wherein resulting amplicons of the sequence of interest will be significantly larger) will produce fewer amplicons than the wild-type sequence of interest or deletion mutations of the sequence of interest.

### Example 2. Quality Control Assessment of Libraries

A quantitative PCR (qPCR) assay was performed for quality control (QC) of libraries. The QC qPCR assay used PrimeStar GXL DNA polymerase (Takara), a long-range polymerase known to be able to amplify long targets (e.g. greater than 30 kb) with high fidelity, to amplify non-nicked template strands. During amplification, the forward primer, specific to the hairpin adapter contained in the library molecules, will extend to the opposite adapter and create a new template strand for the reverse primer only if the template is not disrupted by a nick. In contrast, a signal from a new template strand will not be generated if the polymerase encounters a nick (as shown in FIG. 9).

Control experiments were run to determine how nicks affected Cq values. A qPCR master mix consisted of 0.5 U long-range polymerase (PrimeStar GXL polymerase), a forward and reverse primer each designed to bind to a specific sequence within the hairpin adapters, 1× EvaGreen, 200 μM of each dNTP, lx PrimeStar buffer, and approximately 200 pg/μ0.1 DNA input (input can be decreased to fg range if necessary).

The 20× EvaGreen was diluted to 5× in water and then included on the reaction plate with a standard curve (library with Nextera adapters and P5/P7 amplification primers) that was run with the samples in order to confirm efficient amplification. The following cycling parameters were performed: initial denaturation at 95° C. for 2 minutes, followed by 30 cycles of 95° C. for 30 seconds, 50° C. for 30 seconds, and 68° C. for 15 seconds. Reactions were run in duplicate, and Cq values were averaged.

Table 2 provides a summary of the qPCR mastermix.

TABLE 2
qPCR mastermix

| | | | | Vendor/ |
|---|---|---|---|---|
| Reagent | μl | Final | | Catalog# |
| 1 ng/μl of approximately | 2 | 200 | pg/μl | |
| 10 kb hairpin library | | | | |
| 100 μM forward primer | 0.05 | 500 | nM | IDT |
| 100 μM reverse primer | 0.05 | 500 | nM | IDT |
| 5X EvaGreen | 1.6 | 0.8X | | Biotium, |
| | | | | #31000 |
| 2.5 mM each dNTP | 0.8 | 200 μM each | | Takara |
| | | | | R050A |
| 5X Primestar buffer | 2 | 1X | | Takara |
| | | | | R050A |
| PrimeStar GXL | 0.2 | 0.025 | U/μl | Takara |

| | | |
|---|---|---|
| polymerase (1.25 U/μl) | | R050A |
| H₂O | 3.3 | |
| Total volume | 10 | |

EvaGreen® Dye and EvaGreen® Plus Dye are green fluorescent nucleic acid dyes that are essentially nonfluorescent by themselves, but which become highly fluorescent upon binding to dsDNA. Accordingly, EvaGreen can be used for digital PCR and isothermal amplification applications.

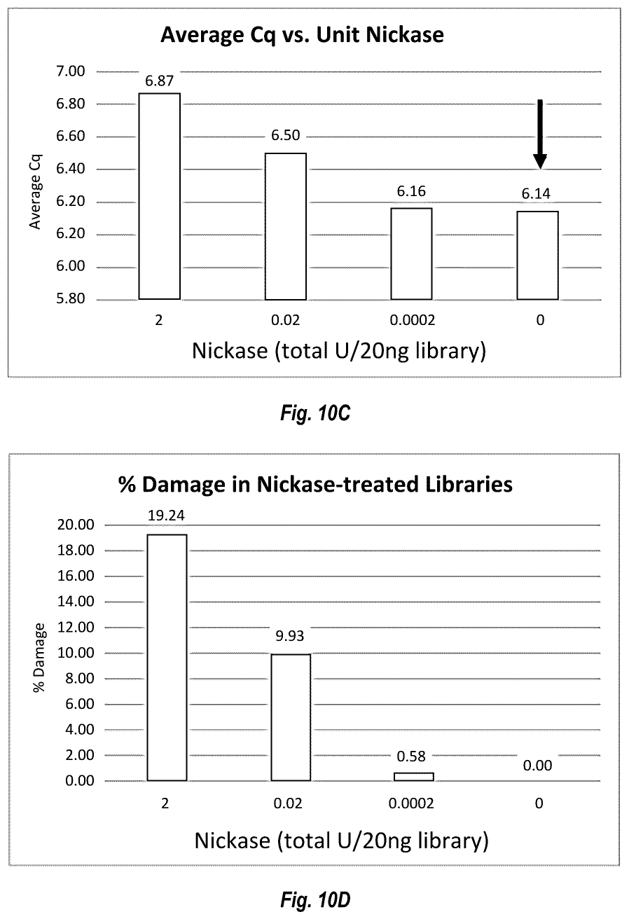

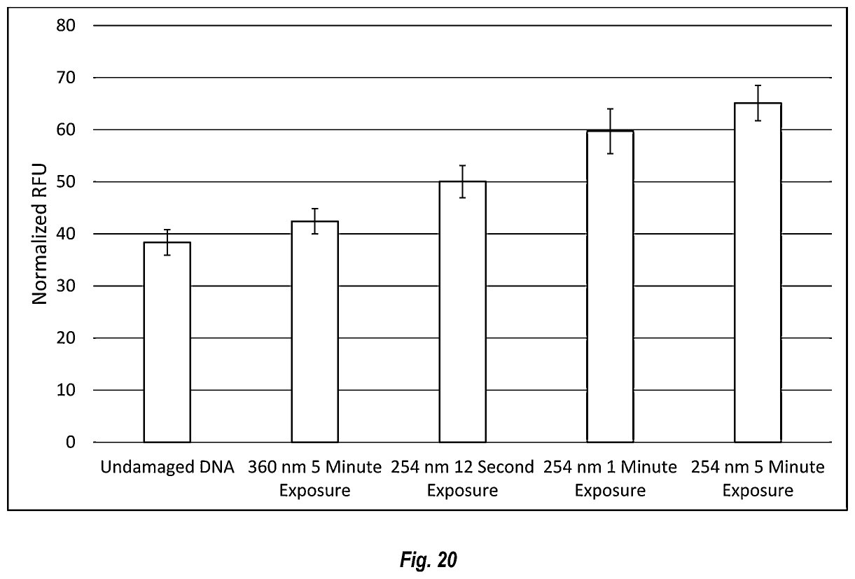

Nickase treatment caused a dose-dependent increase in DNA damage and in average Cq, for both 10 ng libraries (FIGS. 10A and 10B) and 20 ng libraries (FIGS. 10C and 10D). These results indicate that qPCR results from this QC assay will generate lower Cqs for higher quality libraries, and higher Cqs for damaged libraries (e.g., those comprised of library molecules containing nicks).

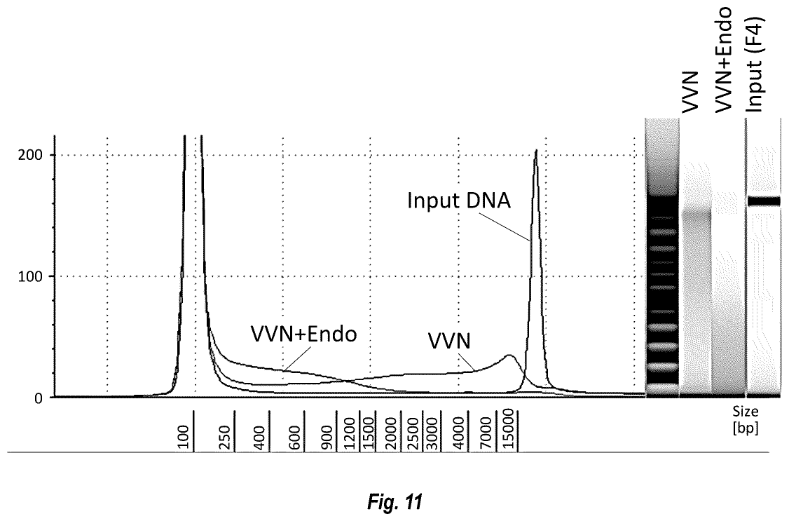

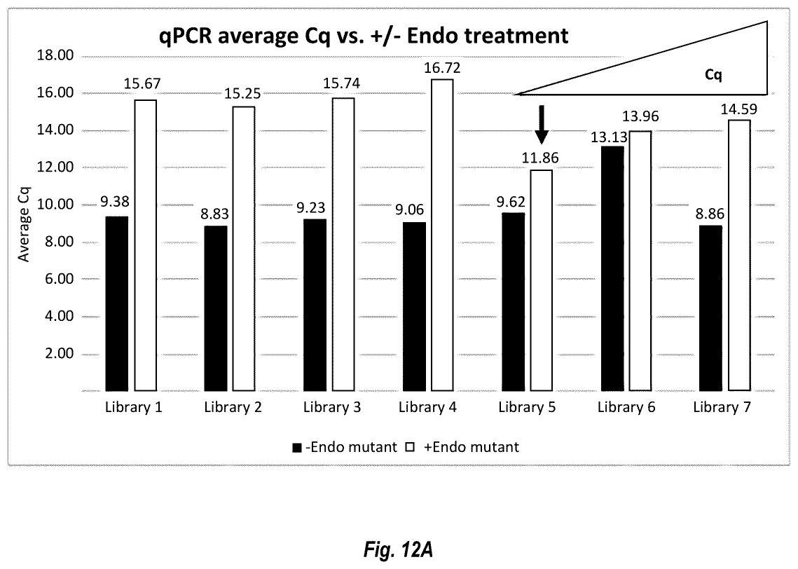

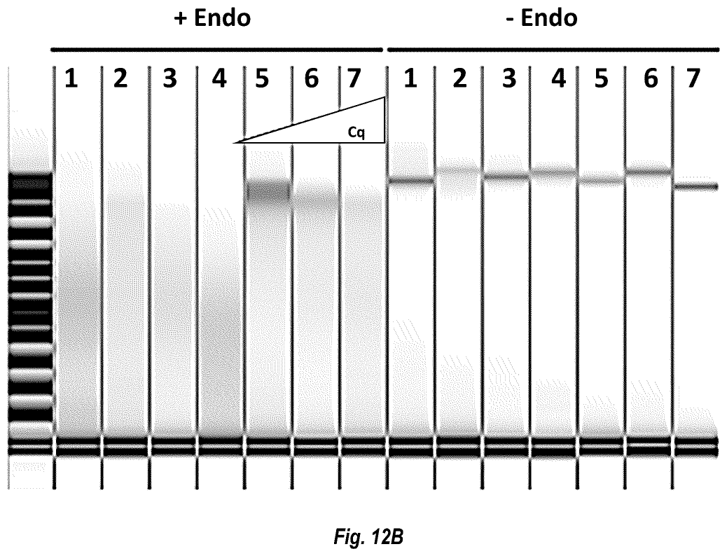

Similar results were seen following endonuclease treatment (FIG. 11 and FIGS. 12A and 12B) to prepare double-stranded breaks from nicks using a combination of Vibrio vulnificus nuclease (VVN, a non-specific nuclease) and a T7 endonuclease mutant. Thus, preparing double-stranded breaks from nicked templates, resulting in the separation of the primer sequences required for amplification, further demonstrates the QC assay is capable of identifying libraries of insufficient quality.

### Example 3. Quality Control of SMRTbell Libraries

FIGS. 13A-15C show additional experiments with SMRTbell libraries, which contain hairpin adapters at both ends of double-stranded fragments, using methods described in Example 2. These analyses across different libraries confirm that total sequencing output consistently increases for libraries with lower Cq values. In other words, there was a strong correlation between qPCR results in the QC step and the measured total sequencing output (i.e., gigabases sequenced). Generally, libraries having a lower Cq value in the QC assay had higher total sequencing output. For example, a percentage P1 variation between 39%-67% was seen for libraries with a Cq value of approximately 3 in the QC assay, compared to 17% when the Cq value exceeded 9 (FIGS. 13A-13C). Library 8 is noted as an outlier to this relationship.

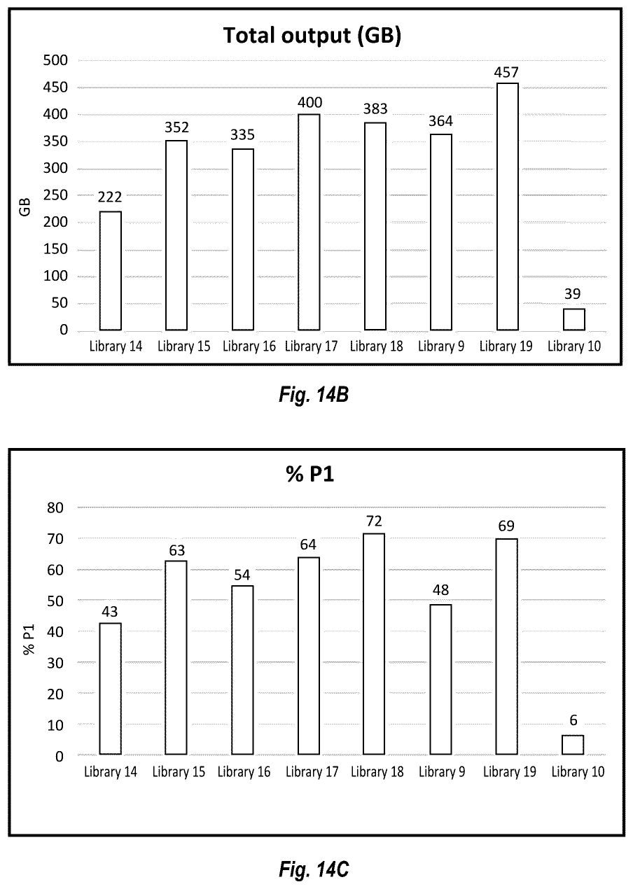

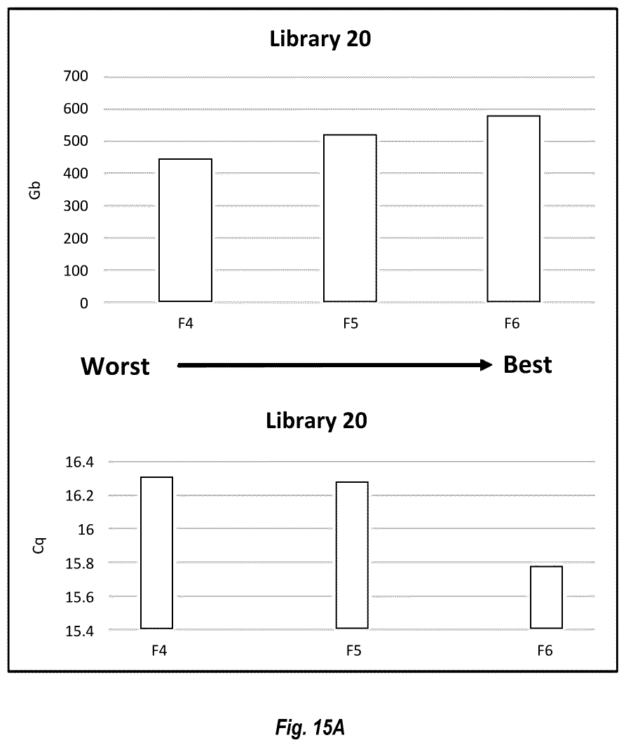

Further, data in FIGS. 14A-14C indicate that Cq values in the range of 3-4 generated approximately 366 gigabases on average. In contrast, Library 10 was predicted to be a poor performer based on its QC value of over 6 (FIG. 14A), and the sequencing results showed a relatively poor total output and percentage P1 (FIGS. 14B and 14C). Thus, the QC assay was able to predict a library that would have poor sequencing performance. Generally, a relationship was seen that the lower the average Cq for a library, the higher the percentage P1, though this was not true for Library 14 (corresponding to Library 8 in FIGS. 13A-13C).

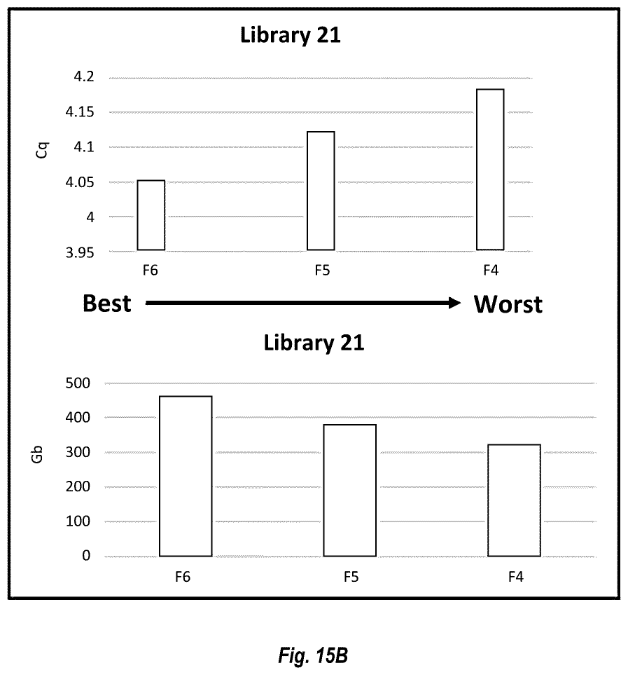

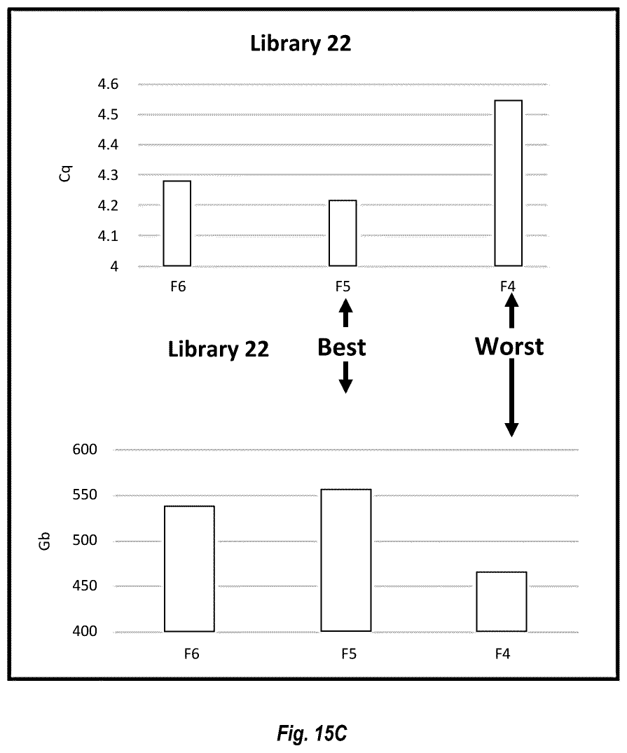

FIGS. 15A-15C similar show the best total sequencing output (gigabases) was seen for library fractions (i.e., different fractions prepared from the same library, such as F4, F5, and F6) with lower Cq values in the QC assay, in comparison to library fractions with higher Cq values.

Thus, the present QC method is a valuable tool for making decisions about sequencing (or not sequencing) individual libraries. Such a QC method is particularly valuable as libraries may vary in quality in ways that a user cannot predict based on existing QC methods alone. For example, pipetting force used with one sample may cause degradation that is not seen with other libraries generated by the same user. Only a method that can assess the quality of libraries that have already been produced can control for random variables that impact on the quality of sequencing data. Thus, one skilled in the art may use initial experiments to generate a range of desired Cq values, based on the specific libraries being used, that can be used to select libraries for sequencing using the QC method.

### Example 4. Measuring DNA Damage Using Fluorescence

A user may also want to measure DNA damage using fluorescence. For example, a user may want to measure DNA damage before preparing a library to ensure that the level of DNA damage in a sample is acceptable. For example, a user may want to use a method of quantifying DNA damage that is flexible to use on genomic DNA or cDNA before library preparation or on a library that has already been prepared. However, current assays containing both fluorescently labeled nucleotides and proteins often suffer from high nonspecific binding of unincorporated fluorescent nucleotides.

The present assay was developed to improve the signal-to-noise ratio of the fluorescent quantification. This method employs both a shrimp alkaline phosphatase (SAP) digestion and a SPRI (carboxylate bead) binding/elution step to significantly reduce nonspecific binding. Depending on user preference, cellulose beads may be used in place of carboxylate beads and calf intestinal alkaline phosphate may be used in place of SAP in any of the methods described.

FIG. 16 outlines the present method, which incorporates a DNA repair step (in this example with Bst polymerase and Taq ligase) in the presence of fluorescently labeled dNTPs, followed by treatment with SAP and two steps of SPRI bead-based purification. The treated sample comprising repaired DNA is then measured to determine the amount of fluorescence.

Initial experiments tested different conditions for reducing nonspecific binding of dNTPs. FIG. 17 shows that with a single SPRI bead-based purification, SAP treatment of sheared and genomic DNA (gDNA) substantially reduced nonspecific binding of fluorescent nucleotides as compared to an assay without SAP treatment. In other words, a bead-based purification step together with SAP treatment reduced non-specific fluorescence.

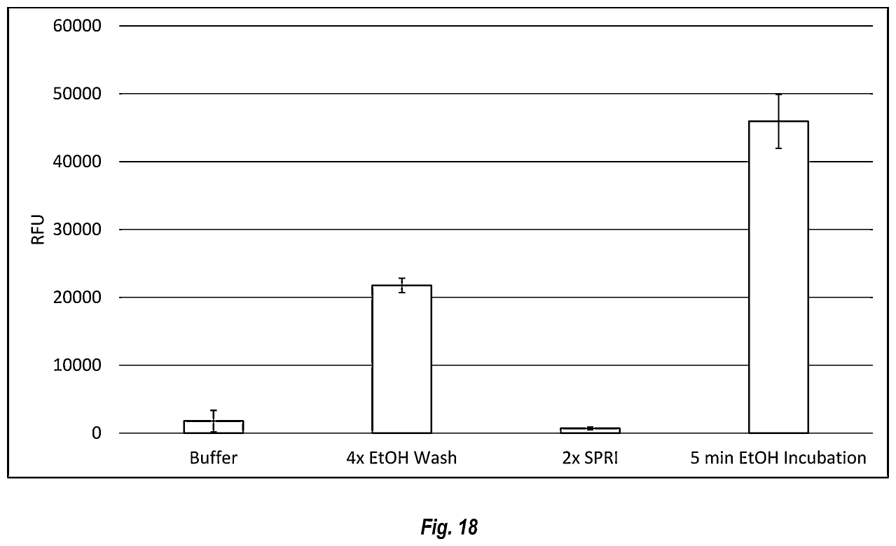

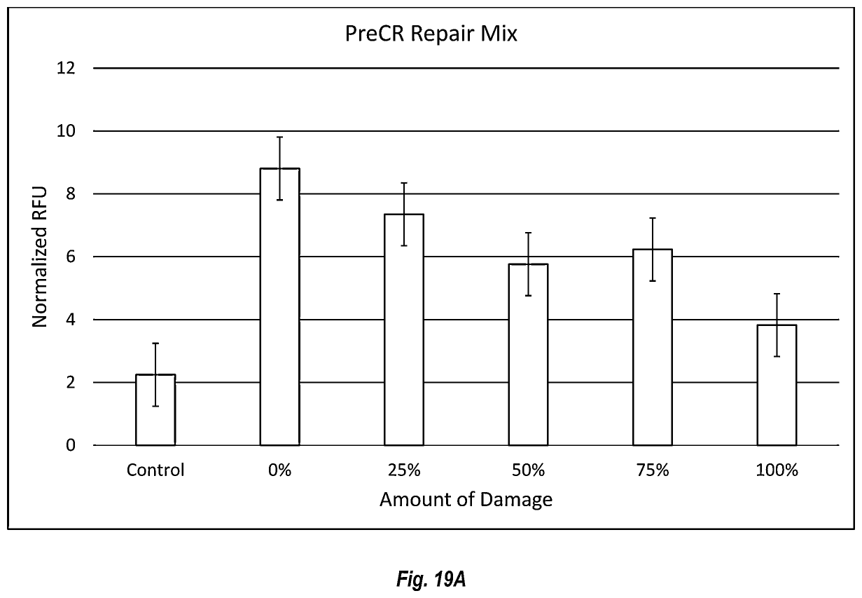

Further, FIG. 18 shows that a second SPRI bead-based purification step dropped nonspecific binding of fluorescent nucleotides to the level comparable to buffer. Such a low background is important for accurately measuring small amounts of DNA damage (i.e., when a low percentage of nucleotides in a DNA are damaged).

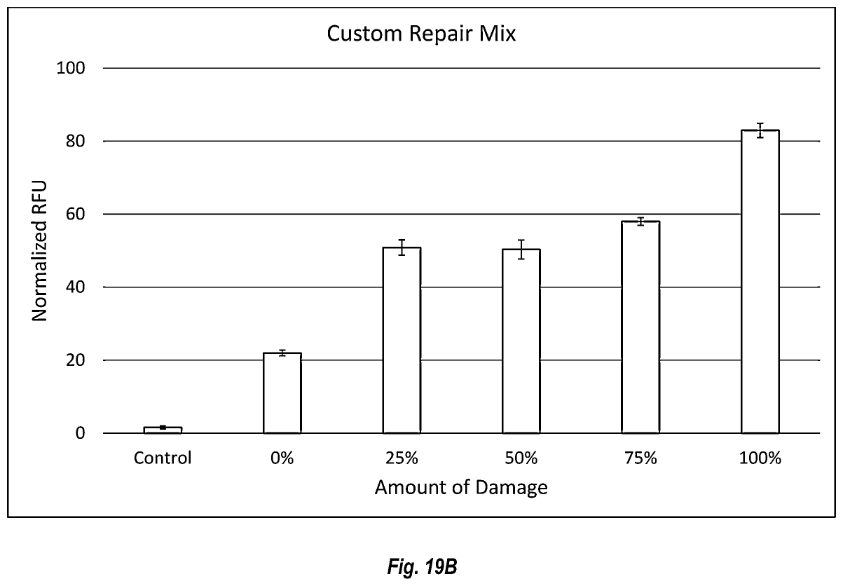

Based on initial experiments, two steps of SPRI bead-based purification were performed after SAP treatment in further experiments. A comparison was made of efficacy of a commercially available repair mix versus an in-house DNA repair enzyme mix with the present method. PreCR Repair Mix (NEB) was compared to a custom repair mix of Taq ligase (40 U), Bst polymerase large fragment (8 U), and T4 PDG (1 U) with the present protocol. As shown in FIG. 19A, while the PreCR Mix did not exhibit expected fluorescence increases as damage of samples increased, the custom repair mix exhibited these expected increases. PreCR Mix samples also had larger standard deviations and low signal, and such inconsistencies can also be found in literature from groups optimizing DNA damage repair formulations. In contrast, the custom repair enzyme mix using present method had low standard deviation and higher signal-to-noise ratio (FIG. 19B).

The present method with a custom mix of DNA repair enzymes determined by the user also adds flexibility to the workflow because the user is able to choose which repair enzymes to utilize in the assay. For example, the present assay can be designed to detect different types of damage in DNA by utilizing different DNA damage repair enzymes. Incorporating the T4 pyrimidine dimer glycosylase (T4 PDG) enzyme in a DNA repair enzyme mix can allow for the repair and subsequent detection of damage caused by UV irradiation, such as thymine dimers. As shown in FIG. 20, a method using a DNA repair enzyme mixture comprising Taq ligase, Bst polymerase, and T4 PDG (a UV-damage specific repair enzyme) could assess UV-induced DNA damage. As the amount of UV light and exposure time increased, DNA damage as measured by the present assay also increased, showing the ability of the present assay to measure DNA damage over a broad range.

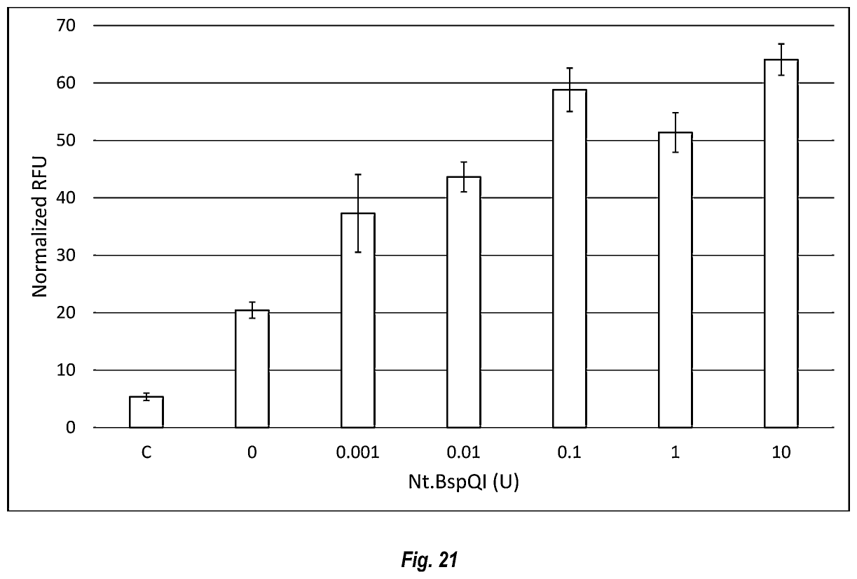

FIG. 21 further shows that when a DNA sample is exposed to different amount of a nicking enzyme (Nt.BspQI), the fluorescent signal of the DNA damage measurement increased. Thus, the present assay can sensitively measure the amount of nicked DNA over a broad range.

If desired by the user, incorporating uracil DNA glycosylase (UDG) and an apurinic or apyrimidinic site lyase and/or formamidopyrimidine DNA glycosylase (FPG) and an apurinic or apyrimidinic site lyase in the enzyme repair mix can allow for the repair and subsequent detection of uracil or oxidized bases, respectively.

The modularity of this assay makes it a flexible and customizable tool for detecting different types of damage in double-stranded DNA, based on the activity and specificity of the enzymes used.

### Example 5. Measuring DNA Damage Using Fluorescence

Based on initial experiments, an exemplary assay protocol was developed for use with a DNA repair enzyme mix comprising Taq ligase, Bst polymerase, and T4 PDG. Table 3 provides reagents for use in this assay, while Table 4 provides dNTP master mix contents, and Table 5 provides DNA damage assay contents.

TABLE 3
Reagents for Measuring DNA Damage

| Material | Supplier | Part Number |
|---|---|---|
| MilliQ Water | — | — |
| 200 Proof Ethanol | Sigma Aldrich | E7023 |
| 0.2 mL Strip Tubes | USA Scientific | 1402-4708 |
| AMPure PB Beads | Pacific | 100-265-900 |
| | Biosciences | |
| 10x ThermoPol Buffer | New England | B9004S |
| | Biolabs | |
| 100x NAD+ | New England | B9007S |
| | Biolabs | |
| Alexa Fluor 546-14-dUTP, 1 mM | Thermo Fisher | C11401 |
| Alexa Fluor 555-aha-dCTP, 1 mM | Thermo Fisher | A32770 |
| dGTP, 100 mM | Promega | U1330 |
| dATP, 100 mM | Promega | U1330 |
| Resuspension Buffer | Illumina | — |
| Bst Polymerase, Large | New England | M0275S |
| Fragment (8 U/ul) | Biolabs | |
| Taq Ligase (40 U/ul) | New England | M0208S |
| | Biolabs | |
| T4 PDG (10 U/ul) | New England | M0308S |
| | Biolabs | |
| Shrimp Alkaline | New England | M0371S |
| Phosphatase (1 U/ul) | Biolabs | |
| Black 96-well half area plates | Corning | 3694 |
| Qubit 1x dsDNA High Sensitivity kit | Thermo Fisher | Q33231 |
| 0.5 mL Qubit Tubes | Thermo Fisher | Q32856 |

TABLE 4
10 μM dNTP Master Mix contents

| Component | Volume |
|---|---|
| Alexa Fluor 546-14-dUTP, 100 | 0.25 μl |
| uM | |
| Alexa Fluor 555-aha-dCTP, 100 | 0.25 μl |
| uM | |
| dGTP, 100 uM | 0.25 μl |
| dATP, 100 uM | 0.25 μl |
| MilliQ Water | 9 μl |
| Total Volume | 10 μL |

TABLE 5
DNA Damage Assay Contents

| Component | Volume |
|---|---|
| MilliQ Water | 2.4 μL |
| 10x ThermoPol buffer | 1 μL |
| dNTP Master Mix, 10 μM | 2.5 μl |
| Bst, Large Fragment (8 U/μl) | 1 μL |
| Taq Ligase (40 U/μl) | 1 μL |
| T4 PDG (10 U/μl) | 0.1 μl |
| gDNA (200 ng) | 2 μl |
| Total Volume | 10 μL |

A representative assay protocol can be performed as follows:
1. Prepare dNTP dilutions and dNTP master mix as described in Tables 4 and 5. Place on ice.
2. Quantify sample and control gDNA using Qubit. Dilute gDNA to 100 ng/μ0.1 and place on ice.
3. Prepare assay mix in a strip tube on ice, in duplicate per sample, and gently pipette to mix. Incubate at 37° C. for 30 minutes in a thermocycler with heated lid.
4. After 30 minutes, remove from thermocycler and add 1 μl of shrimp alkaline phosphatase (SAP) to each sample. Gently pipette to mix and incubate at 37° C. for 60 minutes in a thermocycler with a heated lid.
5. After incubation, dilute to 100 μl with resuspension buffer (RSB). Vortex AMPure PB (SPRI) beads to mix and add 100 μl of SPRI beads. Pipette to mix and gently shake at room temperature for 15 minutes.
6. Magnetize beads using a benchtop magnetic rack and wash samples twice with 100 μl of 80% ethanol, without disturbing the bead pellet. Make sure to spin down and aspirate all ethanol completely after the second wash.
7. Resuspend beads in 100 μl of RSB. Gently shake at room temperature for 15 minutes.
8. Magnetize beads using a benchtop magnetic rack and aspirate supernatant into a new strip tube.
9. Optionally repeat SPRI cleanup (steps 5-8).
10. Prepare a 100 μl standard curve using AF-546 dUTP in RSB, starting at 5 nM and decreasing in concentration by half. (5 nM, 2.5 nM, 1.25 nM, 625 pM, 312 pM, 156 pM, 78 pM, and 39 pM)
11. Pipette 45 μl of each purified sample in duplicate into a 96-well plate. Pipette 45 μl of the standard curve in duplicate into the 96-well plate.
12. Place the plate into the plate holder of the Cytation 5 multi-mode reader (Agilent). Select Alexa Fluor 546 as the fluorophore and measure the fluorescence of the sample and standard curve in a single read.
13. Dilute the leftover samples and control 1:10 in RSB and quantify the recovered DNA with Qubit. Using the standard curve, calculate the molecules of dye incorporated into the DNA. Divide # of dye molecules by mass of recovered gDNA to determine normalized # of dye molecules.

One skilled in the art can use this representative protocol with a DNA repair enzyme mix of their preference to evaluate DNA damage in a sample.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +1-5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A pool of nucleic acid standards of different lengths, wherein the nucleic acid standards comprise a unique molecular identifier (UMI) and:
a. a 5′ universal oligonucleotide, wherein the 5′ universal oligonucleotide is the same for all standards;
b. a 3′ universal oligonucleotide, wherein the 3′ universal oligonucleotide is the same for all standards; and
c. at least one region between the UMI and the 5′ universal oligonucleotide and/or between the UMI and the 3′ universal oligonucleotide;
wherein the length of the at least one region determines the length of the standard.

2. The pool of standards of claim 1, wherein the pool further comprises a further nucleic acid standard that comprises a UMI and:
a. a 5′ universal oligonucleotide, wherein the 5′ universal oligonucleotide is the same for all standards; and
b. a 3′ universal oligonucleotide, wherein the 3′ universal oligonucleotide is the same for all standards;
wherein the further nucleic acid standard does not comprise at least one region between the UMI and the 5′ universal oligonucleotide or between the UMI and the 3′ universal oligonucleotide.

3. The pool of standards of claim 1, wherein the at least one region between the UMI and the 5′ universal oligonucleotide and/or between the UMI and the 3′ universal oligonucleotide comprise 0.2 kb-10 kb.

4. The pool of standards of claim 1, wherein the 5′ universal oligonucleotide and/or the 3′ universal oligonucleotide each comprise an amplicon amplified from a sequence of interest.

5. The pool of standards of claim 1, wherein the at least one region between the UMI and the 5′ universal oligonucleotide and/or between the UMI and the 3′ universal oligonucleotide each comprise an amplicon amplified from a sequence of interest.

6. The pool of standards of claim 1, wherein the least one region between the UMI and the 5′ universal oligonucleotide and/or between the UMI and the 3′ universal oligonucleotide each comprise an arbitrary sequence.

7. A pool of nucleic acid standards of different lengths, wherein the nucleic acid standards comprise a UMI and:
a. a 5′ partially overlapping oligonucleotide, wherein the 5′ partially overlapping oligonucleotide is identical over at least a portion of its sequence for all the standards; and/or
b. a 3′ partially overlapping oligonucleotide, wherein the 3′ partially overlapping oligonucleotide is identical over at least a portion of its sequence for all the standards;
wherein the lengths of the 5′ partially overlapping oligonucleotide and/or the 3′ partially overlapping oligonucleotide determines the length of the standard.

8. The pool of standards of claim 1, wherein the standards are double-stranded.

9. The pool of standards of claim 1, wherein the standards comprise double-stranded DNA.

10. The pool of standards of claim 1, wherein each standard comprises a different UMI.

11. The pool of standards of claim 1, wherein the UMIs comprised in the pool of standards are a random set of sequences comprising 16-20 base pairs.

12. The pool of standards of claim 11, wherein the UMIs comprised in the pool of standards are a random set of sequences comprising 18 base pairs.

13. The pool of standards of claim 1, wherein the pool of standards comprises 1×10¹⁰ or greater, 10×10¹⁰ or greater, or 100×10¹⁰ or greater standards, wherein each standard comprises a different UMI.

14. The pool of standards of claim 1, wherein the number of standards in the pool is greater than the number of amplicons generated by an amplification reaction.

15. A pool of standards, wherein:
a. at least a first portion of the standards are from claim 1; and
b. at least a second portion of the standards are a pool of nucleic acid standards of different lengths, wherein the nucleic acid standards comprise a UMI and:
i. a 5′ partially overlapping oligonucleotide, wherein the 5′ partially overlapping oligonucleotide is identical over at least a portion of its sequence for all the standards; and/or
ii. a 3′ partially overlapping oligonucleotide, wherein the 3′ partially overlapping oligonucleotide is identical over at least a portion of its sequence for all the standards, wherein the lengths of the 5′ partially overlapping oligonucleotide and/or the 3′ partially overlapping oligonucleotide determines the length of the standard.

16. A method of generating a pool of nucleic acid standards comprising:
a. providing multiple copies of at least one sequence of interest comprising nucleic acids;
b. providing a collection of oligonucleotides each comprising a UMI;
c. providing a collection of insertion oligonucleotides of varying lengths; and
d. ligating at least one sequence of interest of (a), at least one oligonucleotide comprising a UMI of (b), and at least one insertion amplicon of (c) to produce multiple nucleic acid standards of the pool of nucleic acid standards.

17. A method of generating a pool of nucleic acid standards comprising:
a. providing multiple copies of at least one sequence of interest comprising nucleic acids;
b. providing a collection of oligonucleotides each comprising a UMI; and
c. ligating at least one sequence of interest of (a) and at least one oligonucleotide comprising a UMI of (b).

18. A method of normalizing amplicon size bias comprising:
a. combining a sample comprising a target nucleic acid with a pool of nucleic acid standards of different lengths, wherein each standard comprises a UMI;
b. amplifying the standards and amplicons of a sequence of interest comprised in the target nucleic acid;
c. sequencing the standards and the amplicons of the sequence of interest to generate sequencing data;
d. determining a bias profile based on amplicon size using sequencing data from the standards; and
e. normalizing amplicon size bias using the bias profile.

19. A method of determining the presence of DNA damage in a library comprising one or more library molecule, wherein each library molecule comprises a double-stranded DNA insert with a hairpin adapter at each end of the insert, comprising:
a. denaturing the first stand and second strand of the double-stranded DNA inserts comprised in library molecules;
b. annealing a forward primer and a reverse primer to library molecules;
c. amplifying to produce library amplicons; and
d. assessing the presence of DNA damage based on the number of library amplicons produced.

20. A method of quantifying DNA damage in a sample comprising DNA using fluorescence comprising:
a. combining:
i. an aliquot of a sample comprising DNA,
ii. one or more DNA repair enzyme; and
iii. dNTPs, wherein one or more dNTP is fluorescently labeled;
b. preparing repaired DNA;
c. dephosphorylating the phosphates from dNTPs;
d. binding the repaired DNA to carboxylate or cellulose beads;
e. eluting the bound repaired DNA from the carboxylate or cellulose beads with a resuspension buffer; and
f. measuring fluorescence of the repaired DNA to determine the amount of DNA damage.
